(19) United States
(12) Patent Application Publication
(10) Pub. No.: US 2023/0313210 A1
(43) Pub. Date: Oct. 5, 2023

(54) ENGINEERED ORAL BACTERIA AND USES THEREOF

(71) Applicant: ARIZONA BOARD OF REGENTS ON BEHALF OF THE UNIVERSITY OF ARIZONA, Tucson, AZ (US)

(72) Inventors: Eric H. Lyons, Tucson, AZ (US); Scott Zentack, Tucson, AZ (US); David A. Baltrus, Tucson, AZ (US)

(21) Appl. No.: 18/024,647

(22) PCT Filed: Sep. 3, 2021
(86) PCT No.: PCT/US2021/049114

§ 371 Date: Mar. 3, 2023

Related U.S. Application Data
(60) Provisional application No. 63/074,802, filed on Sep. 4, 2020.

(51) Int. Cl. C12N 15/78; C12N 9/10; C12N 9/88; C12N 9/90; A61K 8/99; A61Q 11/00; A23K 10/16; A23K 50/40

(52) U.S. Cl. CPC C12N 15/78; C12N 9/1007; C12N 9/88; C12Y 402/99021; C12N 9/90; C12Y 504/04002; C12N 9/1029; C12Y 203/01084; A61K 8/99; A61Q 11/00; A23K 10/16; A23K 50/40

(57) ABSTRACT

Described herein are engineered bacteria that can produce a volatile compound and formulations thereof. Also described herein are methods of improving the breath odor of a mammal by administering the engineered bacteria or formulation thereof to the oral cavity of the mammal.

### CROSS-REFERENCE TO RELATED APPLICATIONS

This application claims the benefit of and priority to co-pending U.S. Provisional Patent Application No. 63/074,802, filed on Sep. 4, 2020, entitled “ENGINEERED ORAL BACTERIA AND USES THEREOF,” the contents of which is incorporated by reference herein in its entirety.

### SEQUENCE LISTING

This application contains a sequence listing filed in electronic form as an ASCII.txt file entitled UAZ-0115WP created on Sep. 3, 2021, and having the size of 6,415,931 bytes. The content of the sequence listing is incorporated herein in its entirety.

### TECHNICAL FIELD

The subject matter disclosed herein is generally directed to engineered bacteria containing gene(s) to express one or more proteins associated with the production of volatile compounds.

### BACKGROUND

Dogs and cats can have bad breath. Not only can this be a nuisance for their human companions, the offensive odor has roots in an unhealthy microflora. Although treats, chews, mouthwashes, and toothpastes exist for pets, most fail to have long term efficacy and do not mask the odor for sustained periods of time. While some aid in inhibiting or preventing gum disease and tooth decay by mechanical action (e.g., chews), the market is devoid of options that assist in establishing the healthy commensal oral microflora. As such, there exists a need for compositions, techniques, and/or methods that can address halitosis in at least companion animals.

Citation or identification of any document in this application is not an admission that such a document is available as prior art to the present invention.

### SUMMARY

Described in certain aspects is an engineered bacterium or population thereof, wherein the engineered bacterium containing one or more polynucleotides that each encode one or more enzymes capable of catalyzing one or more reactions to produce a volatile compound, one or more polypeptides produced therefrom, or both, and wherein the engineered bacterium is of a strain that is a commensal bacterium strain found in the oral cavity of a mammal, optionally a canine or a feline.

In certain example embodiments, the one or more enzymes is/are
a. a methyltransferase that catalyzes the production of methyl salicylate from salicylic acid;
b. an isochorismate synthase;
c. an ischorisomate-pyruvate lyase;
d. is an alcohol acetyl transferase that is capable of condensing isoamyl alcohol and acetyl CoA into isoamyl acetate; or
e. any combination thereof.

In certain example embodiments, the one or more polynucleotides each encode a methyltransferase that catalyzes the production of methyl salicylate from salicylic acid, an isochorismate synthase, an ischorisomate-pyruvate lyase, or any combination thereof.

In certain example embodiments, the volatile compound produced has a mint odor.

In certain example embodiments, the one or more polynucleotides each comprise a PCHBA gene or mRNA, a BSMT1 gene or mRNA, or any combination thereof.

In certain example embodiments, at least one of the one or more polynucleotides encodes an alcohol acetyltransferase that is capable of condensing isoamyl alcohol and acetyl CoA into isoamyl acetate.

In certain example embodiments, the engineered bacterium or population thereof produces a fruity odor. In certain example embodiments, the fruity odor is a banana odor or a pear odor.

In certain example embodiments, at least one of the one or more polynucleotides includes an ATF gene or mRNA, an BAT2 gene or mRNA, a THI3 gene or mRNA, or any combination thereof.

In certain example embodiments, the ATF gene or mRNA is an ATF1 gene or mRNA or an ATF2 gene or mRNA.

In certain example embodiments, one or more of the one or more polynucleotides each comprise a gene or mRNA independently selected from a BSTM1 gene, a BSTM1 mRNA, an ATF gene, an ATF mRNA, a PCHBA gene, a PCHBA mRNA, a BAT2 gene, a BAT2 mRNA, a THI3 gene, a THI3 mRNA, or any combination thereof.

In certain example embodiments, the ATF gene or ATF mRNA is an ATF1 gene or mRNA or an ATF2 gene or mRNA.

In certain example embodiments,
a. the BSTM1 gene or mRNA has a polynucleotide sequence that is about 90-100 percent identical to any one of SEQ ID NO: 2, 8, 11, or 13;
b. the PCHBA gene or mRNA has a polynucleotide sequence that is about 90-100 percent identical to SEQ ID NO: 1 or 10;
c. the ATF gene or mRNA has a polynucleotide sequence that is about 90-100 percent identical to SEQ ID NO: 49 or 51;
d. the BAT2 gene or mRNA has a polynucleotide sequence that is about 90-100 percent identical to SEQ ID NO: 53;
e. the THI3 gene or mRNA has a polynucleotide sequence that is about 90-100 percent identical to SEQ ID NO: 55;
f. or any combination thereof

In certain example embodiments, one or more of the one or more polynucleotides has a sequence that is about 40-100% identical to any one of SEQ ID NOs.: 1, 2, 8, 10-11, 13, 49, 51, 53, or 55 or any region therein of at least 20 nucleotides.

In certain example embodiments, one or more of the one or more polynucleotides encodes a polypeptide having a sequence that is about 40 to 100% identical to any one of SEQ ID NOS.: 3-7, 9, 50, 52, 54, or 56.

In certain example embodiments, the engineered bacterium or population thereof further contains one or more expression vectors, wherein the one or more expression vectors comprise the one or more polynucleotides.

In certain example embodiments, the engineered bacterium is from genus selected from Escherichia, Pseudomonas, Brevundimonas, Xanthomonas, Deinococcus, or Pedobacter.

In certain example embodiments, the engineered bacterium is an isolate of a commensal canine oral bacterium or population thereof.

In certain example embodiments, the commensal canine oral bacterium or population thereof is of the genus Pseudomonas.

In certain example embodiments, the engineered bacterium is an isolated commensal canine oral bacterium or population thereof having a genome having sequence that is 90-100 percent identical to any one or more of SEQ ID NOs: 12 or 14-48.

Described in certain aspects is an expression vector capable of expressing a BSTM1 polypeptide, a PCHB polypeptide, a PCHA polypeptide, an ATF1 polypeptide, an ATF2 polypeptide, BAT2 polypeptide, a THI3 polypeptide or any combination thereof having:
a. a BSTM1 polynucleotide having a sequence that is about 90-100 percent identical to any one of SEQ ID NO: 2, 8, 11, or 13;
b. a PCHBA polynucleotide having a sequence that is about 90-100 percent identical to SEQ ID NO: 1 or 10;
c. an ATF polynucleotide having a sequence that is about 90-100 percent identical to SEQ ID NO: 49 or 51;
d. the BAT2 polynucleotide having a sequence that is about 90-100 percent identical to SEQ ID NO: 53;
e. the THI3 polynucleotide having a sequence that is about 90-100 percent identical to SEQ ID NO: 55;
f. or any combination thereof.

In certain example embodiments, (a), (b), (c), (d), (e), or (0 of the expression vector is/are operatively coupled one or more regulatory elements.

In certain example embodiments, the expression vector includes a sequence that is about 90-100 percent identical to SEQ ID NO: 13.

Described in certain aspects is a formulation having an engineered bacterium as described herein, an expression vector of as described herein, or both, optionally wherein the engineered bacterium contains the expression vector and a liquid, semi-solid, or solid carrier.

In certain example embodiments, the formulation is a foodstuff suitable for a mammal. In certain example embodiments, the mammal is a non-human animal. In certain example embodiments, the mammal is a canine or a feline.

In certain example embodiments, the formulation is a liquid solution or semi-solid suitable for administration to the oral cavity of a mammal. In certain example embodiments, the mammal is a non-human animal. In certain example embodiments, the mammal is a canine or a feline.

In certain example embodiments, the formulation is a formed object that is configured to be chewed on by a mammal. In certain example embodiments, the mammal is a non-human animal. In certain example embodiments, the mammal is a canine or a feline.

Described in certain aspects herein is a method of improving the breath of a mammal, the method including administering an engineered bacterium or population thereof as described herein, an expression vector as described herein, or both, or a formulation thereof to the mammal.

In certain example embodiments, the method further includes allowing the engineered bacterium or population thereof to produce a volatile compound.

In certain example embodiments, the mammal is a non-human animal. In certain example embodiments, the mammal is canine or a feline.

Described in certain aspects herein is an isolated commensal canine oral bacterium or population thereof having: a genome having a sequence that is 90-100 percent identical to any one or more of SEQ ID NOs: 12 or 14-48.

In certain example embodiments, the isolated commensal canine oral bacterium or population thereof is of the genus Pseudomonas.

These and other embodiments, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Genomes in CoGe analyzed using BLAST alignment tool.

FIG. 2—Confirmation that the two scaffolds isolated (termed DOG53 (top) and DOG91 (bottom)).

FIG. 3—Mint plasmid map.

FIG. 4—Comparative genomics demonstrating DOG53 and DOG91 are nearly 100% identical.

FIG. 5—DOG53 and DOG91 have DNA sequence and genome structure divergence.

FIG. 6—Comparison of Dog53 v. Pseudomonas stutzeri strain RCH2.

FIG. 7—Comparison of Pseudomonas stutzeri strain DSM 4166 v Pseudomonas stutzeri strain RCH2 for an example of what two strains from the same species of Pseudomonas looks like.

FIG. 8—GC/MS spectrograph of bacteria supernatant containing methyl salicylate.

FIGS. 9A-9C—Persistence of Dog53 and production of methyl salicylate. (FIG. 9A) Bacteria recovered from dog mouths at 0 min, 30 min, 60 min, 90 min, and 120 min after feeding. (FIG. 9B) Control for GC/MS of pure methyl salicylate. (FIG. 9C) GC/MS of dog saliva showing presences of methyl salicylate.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further embodiment. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” is a sample that contains whole cells and/or live cells, cell debris, cell components, and/or other biologic material and/or molecules. The biological sample can contain (or be derived from) a “bodily fluid”. The biological sample can contain (or be derived from) a plant. The biological sample can be derived from the environment (e.g., water, soil, air, or other environmental component). Bodily fluids include, but are not limited to, is whole blood or component thereof (e.g., plasma, serum, etc.) amniotic fluid, aqueous humour, vitreous humour, bile, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids, cell cultures from environmental samples, and/or the like. Bodily or other fluids may be obtained from an organism, for example by puncture, or other collecting or sampling procedures.

As used herein, “organism”, “host”, “patient”, “individual”, and “subject” refers to any entity composed of at least one cell. An organism can be as simple as, for example, a single isolated eukaryotic cell or cultured cell or cell line, or as complex as a mammal, including a human and non-human animals (e.g., vertebrates, amphibians, fish, mammals, e.g., cats, dogs, horses, pigs, cows, sheep, rodents, rabbits, squirrels, bears, primates (e.g., chimpanzees, gorillas, and humans)). “Subject” may also be a cell, a population of cells, a tissue, an organ, or an organism, and constituents thereof. Tissues, cells and their progeny of a biological entity obtained in vivo or cultured in vitro are also encompassed by this term.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), ribozymes, crRNA, non-coding RNA, long non-coding RNA, engineered guide RNA for a RNA guided nuclease, and/or the like.

As used herein, “DNA molecule” can include nucleic acids/polynucleotides that are made of DNA.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, the term “encode” refers to principle that DNA can be transcribed into RNA, which can then be translated into amino acid sequences that can form proteins. As such, related terms, such as “encoding” in the context of an “encoding polynucleotide” refer to any polynucleotide capable of indirectly or directly being translated into a protein, including but not limited to DNA, cDNA, and mRNA.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “identity,” is a relationship between two or more nucleotide or polypeptide sequences, as determined by comparing the sequences. In the art, “identity” also refers to the degree of sequence relatedness between nucleotide or polypeptide as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H, and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure, unless stated otherwise.

As used herein, “isolated” means separated from constituents, cellular and otherwise, in which the polynucleotide, peptide, polypeptide, protein, antibody, or fragments thereof, bacteria, are normally associated with in nature. A non-naturally occurring polynucleotide, peptide, polypeptide, protein, antibody, or fragments thereof, or bacteria do not require “isolation” to distinguish it from its naturally occurring counterpart.

As used herein, “mammal,” for the purposes of treatments, refers to any animal classified as a mammal, including human, domestic and farm animals, nonhuman primates, and zoo, sports, or pet animals, such as, but not limited to, dogs, horses, cats, and cows.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules including DNA and RNA that may be single-stranded or, more typically, double-stranded or a mixture of single- and double-stranded regions. In addition, polynucleotide as used herein refers to triple-stranded regions including RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs having unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “nucleic acid sequence” and “oligonucleotide” also encompasses a nucleic acid and polynucleotide as defined above.

As used herein, “overexpressed” or “overexpression” can refer to an increased expression level of an RNA or protein product encoded by a gene as compared to the level of expression of the RNA or protein product in a normal or control cell.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same term can be applied to the arrangement of coding sequences and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, “plasmid” as used herein can refer to a non-chromosomal double-stranded DNA sequence including an intact “replicon” such that the plasmid is replicated in a host cell.

As used herein, “protein” as used herein can refer to a molecule composed of one or more chains of amino acids in a specific order. The term protein is used interchangeable with “polypeptide.” The order is determined by the base sequence of nucleotides in the gene coding for the protein. Proteins are required for the structure, function, and regulation of the body's cells, tissues, and organs.

As used herein, the term “recombinant” or “engineered” generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “separated” can refer to the state of being physically divided from the original source or population such that the separated compound, agent, particle, or molecule can no longer be considered part of the original source or population.

As used herein, the term “vector” is used in reference to a vehicle used to introduce an exogenous nucleic acid sequence into a cell and/or from one environment to another. A vector can include a DNA molecule, linear or circular (e.g., plasmids), which includes a segment encoding a polypeptide of interest optionally operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. In some embodiments, vectors, such as expression vectors, can be derived from yeast or bacterial genomic or plasmid DNA, or viral DNA, or may contain elements of both. A vector can be a replicon, such as a plasmid, phage, or cosmid, into which another DNA segment may be inserted so as to bring about the replication of the inserted segment.

As used herein, “plasmid” refers to a circular double stranded DNA loop into which additional DNA segments can be inserted, such as by standard molecular cloning techniques. Plasmids generally have a genetic structure that allows them to exist and replicate within a cell independently of the chromosomes and within the cytoplasm of the cell.

As used herein, term “biodegradable”, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

As used herein, “transforming” when used in the context of engineering or modifying a cell, refers to the introduction by any suitable technique and/or the transient or stable incorporation and/or expression of an exogenous gene in a cell.

As used herein, “selectable marker” refers to a gene whose expression allows one to identify cells that have been transformed or transfected with a vector containing the marker gene. For instance, a recombinant nucleic acid may include a selectable marker operatively linked to a gene or insert of interest and a promoter, such that expression of the selectable marker indicates the successful transformation of the cell with the gene or insert of interest. Examples of selectable markers include, but are not limited to, DNA and/or RNA segments that contain restriction enzyme sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; epitope tags (e.g. FLAG- and His-tags), and, the inclusion of a DNA sequences required for a specific modification (e.g., methylation) that allows its identification. Other suitable markers will be appreciated by those of skill in the art.

As used herein, “promoter” includes all sequences capable of driving transcription of a coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, “polypeptides” or “proteins” are as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “regulatory element” refers to and includes promoters, operons, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990).

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

Various embodiments are described hereinafter. It should be noted that the specific embodiments are not intended as an exhaustive description or as a limitation to the broader embodiments discussed herein. One embodiment described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Dogs and cats can have bad breath. Not only can a nuisance for their human companions, the offensive odor has roots in an unhealthy microflora. Although treats, chews, mouthwashes, toothpastes exist for pets, most fail to have long term duration and thus, do not effectively mask the odor for sustained periods of time.

With that said, described herein are engineered bacteria that express various genes that result in the production of aromatic compounds. The engineered bacteria can be based on bacteria that are part of the commensal oral microbiome of a dog or cat. When present in the oral cavity of a dog or cat the engineered bacteria can produce aromatic compounds and can result in pleasant breath. The engineered bacteria can be incorporated into an article (e.g., chews and toys), treats, foods, oral rinses or pastes that can be taken into the oral cavity of the dog or cat when eating, chewing, playing with and/or otherwise administered to the dog or cat.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Volatile-Producing Bacteria

Described herein are engineered bacteria that can express genes that result in the production of volatile compounds by the bacteria. In some embodiments, the aromatic compounds can have a pleasant odor when smelled by a human. In some embodiments, the engineered bacteria can produce the volatile compound methyl salicylate, which can produce a mint odor. In some embodiments, the engineered bacteria can produce the volatile compound isoamyl acetate, which can produce a pear/banana odor. The engineered bacteria can be engineered express an exogenous PCHA gene, an exogenous PCHB gene, an exogenous ATF gene (e.g., ATF1 and/or ATF2), exogenous BAT2 gene, an exogenous THI3 gene, or an exogenous BSMT1 gene.

Without being bound by theory, gene expression of BSMT1 results in production of a methyltransferase that is involved in the biosynthesis of methyl salicylate from salicylic acid. Engineered bacteria that express the BSMT1 gene can produce methyl salicylate and a mint smell. In some embodiments, the engineered bacteria are also exposed to and/or contain an amount of salicylic acid.

The genes PCHB and PCHA (collectively PCHBA) are part of the pchDCBA operon that Pseudomonas species use to encode enzymes to make pyochelin. PchA is isochorismate synthase, while pchB encodes isochorisomate-pyruvate lyase. PhcA takes chorismate and converts it to isochorismate. PchB turns isochorismate into salicylate and pyruvate. The salicylate can be converted by BSMT into a volatile compound as previously described. Without being bound by theory, expression PCHB and PCHA in the engineered bacteria can provide the enzymatic pathway to produce a substrate for BSMT.

In some embodiments, the PCHB gene or protein expressed therefrom has or includes a sequence that is about 90% to 100% identical to SEQ ID NOs: 1 and/or 10 (gene or mRNA) or SEQ ID NOs: 4 and/or 6 (protein). For example, the PCHB gene or protein can be about 90, 91, 92, 93, 94, 95, 96, 97, 98, 99, 99.1, 99.2, 99.3, 99.4, 99.5, 99.6, 99.7, 99.8, 99.9, to/or about 100% identical to SEQ ID NOs: 1 and/or 10 (gene or mRNA) or SEQ ID NOs: 4 and/or 6 (protein).

In some embodiments, the PCHA gene or protein expressed therefrom has or includes a sequence that is about 90% to 100% identical to SEQ ID NOs: 1 and/or 10 (gene or mRNA) or SEQ ID NOs: 3 and/or 5 (protein). For example, the PCHA gene or protein can be about 90, 91, 92, 93, 94, 95, 96, 97, 98, 99, 99.1, 99.2, 99.3, 99.4, 99.5, 99.6, 99.7, 99.8, 99.9, to/or about 100% identical to SEQ ID NOs: 1 and/or 10 (gene or mRNA) or SEQ ID NOs: 3 and/or 5 (protein).

In some embodiments, the BSMT (e.g., BSMT1) gene or protein expressed therefrom has or includes a sequence that is about 90% to 100% identical to SEQ ID NOs: 2 and/or 8 (gene or mRNA) or SEQ ID NOs: 9 and/or 7 (protein). For example, the BSMT (e.g., BSMT1) gene or protein can be about 90, 91, 92, 93, 94, 95, 96, 97, 98, 99, 99.1, 99.2, 99.3, 99.4, 99.5, 99.6, 99.7, 99.8, 99.9, to/or about 100% identical to SEQ ID NOs: 2 and/or 8 (gene or mRNA) or SEQ ID NOs: 9 and/or 7 (protein).

The ATF gene (e.g., ATF1 and/or ATF2), BAT2 gene, or THI3 gene encode enzymes that can result in production of an alcohol acetyl transferase, which can result in the condensation of isoamyl alcohol and acetyl-CoA to isoamyl acetate. Without being bound by theory, bacteria engineered to express an ATF, BAT2 gene, and/or THI3 gene can produce isoamyl acetate and a corresponding fruity odor (e.g., banana or pear). See e.g., Mason and Dufour. Yeast. 2000.16(14):1287-1298 and Verstrepen et al., Appl Environ Microbiol. 2003. 69(9):5228-5237.

In some embodiments, the ATF gene and/or protein, the BAT gene and/or protein, and/or the THI3 gene or protein is from a Saccharomyces sp., including but not limited to S. cerevisiae.

In some embodiments, the ATF1 gene or protein expressed therefrom has or includes a sequence that is about 90% to 100% identical to GenBank Accession No. NM 001183797.3 (gene or mRNA) (SEQ ID NO: 49) or UniprotID: UniProtKB-P40353 (protein) (SEQ ID NO: 50). For example, the ATF1 gene or protein can be about 90, 91, 92, 93, 94, 95, 96, 97, 98, 99, 99.1, 99.2, 99.3, 99.4, 99.5, 99.6, 99.7, 99.8, 99.9, to/or about 100% identical to GenBank Accession No. NM 001183797.3 (gene or mRNA) (SEQ ID NO: 49) or UniprotID: UniProtKB-P40353 (protein) (SEQ ID NO: 50).

In some embodiments, the ATF2 gene or mRNA or protein expressed therefrom has or includes a sequence that is about 90% to 100% identical to GenBank Accession No. NM_001181306.1 (gene or mRNA) (SEQ ID NO: 51) or UniprotID: UniProtKB-P53296 (protein) (SEQ ID NO: 52). For example, the ATF2 gene or protein can be about 90, 91, 92, 93, 94, 95, 96, 97, 98, 99, 99.1, 99.2, 99.3, 99.4, 99.5, 99.6, 99.7, 99.8, 99.9, to/or about 100% identical to GenBank Accession No. NM 001181306.1 (gene or mRNA) (SEQ ID NO: 51) or UniprotID: UniProtKB-P53296 (protein) (SEQ ID NO: 52).

In some embodiments, the BAT2 gene or mRNA or protein expressed therefrom has or includes a sequence that is about 90% to 100% identical to GenBank Accession No. NM 001183797.3 (gene or mRNA) (SEQ ID NO: 53) or UniprotID: UniProtKB-P47176 (protein) (SEQ ID NO: 54). For example, the BAT2 gene or protein can be about 90, 91, 92, 93, 94, 95, 96, 97, 98, 99, 99.1, 99.2, 99.3, 99.4, 99.5, 99.6, 99.7, 99.8, 99.9, to/or about 100% identical to GenBank Accession No. NM 001181306.1 (gene or mRNA) (SEQ ID NO: 53) or UniprotID: UniProtKB-P47176 (protein) (SEQ ID NO: 54).

In some embodiments, the THI3 gene or protein expressed therefrom has or includes a sequence that is about 90% to 100% SEQ ID NO: 55 (gene or mRNA) or SEQ ID NO: 56 (protein). For example, the BAT2 gene or mRNA or protein can be about 90, 91, 92, 93, 94, 95, 96, 97, 98, 99, 99.1, 99.2, 99.3, 99.4, 99.5, 99.6, 99.7, 99.8, 99.9, to/or about 100% identical to SEQ ID NO: 55 (gene or mRNA) or SEQ ID NO: 56 (protein).

Exemplary ATF1 gene/mRNA
(GenBank Accession No.: NM_001183797.3)
(SEQ ID NO: 49)
(SEQ ID NO: 49)
ATGAATGAAATCGATGAGAAAAATCAGGCCCCCGTGCAAC
AAGAATGCCTGAAAGAGATGATTCAGAATGGGCATGCTCG
GCGTATGGGATCTGTTGAAGATCTGTATGTTGCTCTCAAC
AGACAAAACTTATATCGAAACTTCTGCACATATGGAGAAT
TGAGTGATTACTGTACTAGGGATCAGCTCACATTAGCTTT
GAGGGAAATCTGCCTGAAAAATCCAACTCTTTTACATATT
GTTCTACCAACAAGATGGCCAAATCATGAAAATTATTATC
GCAGTTCCGAATACTATTCACGGCCACATCCAGTGCATGA
TTATATTTCAGTATTACAAGAATTGAAACTGAGTGGTGTG
GTTCTCAATGAACAACCTGAGTACAGTGCAGTAATGAAGC
AAATATTAGAAGAATTCAAAAATAGTAAGGGTTCCTATAC
TGCAAAAATTTTTAAACTTACTACCACTTTGACTATTCCT
TACTTTGGACCAACAGGACCGAGTTGGCGGCTAATTTGTC
TTCCAGAAGAGCACACAGAAAAGTGGAAAAAATTTATCTT
TGTATCTAATCATTGCATGTCTGATGGTCGGTCTTCGATC
CACTTTTTTCATGATTTAAGAGACGAATTAAATAATATTA
AAACTCCACCAAAAAAATTAGATTACATTTTCAAGTACGA
GGAGGATTACCAATTATTGAGGAAACTTCCAGAACCGATC
GAAAAGGTGATAGACTTTAGACCACCGTACTTGTTTATTC
CGAAGTCACTTCTTTCGGGTTTCATCTACAATCATTTGAG
ATTTTCTTCAAAAGGTGTCTGTATGAGAATGGATGATGTG
GAAAAAACCGATGATGTTGTCACCGAGATCATCAATATTT
CACCAACAGAATTTCAAGCGATTAAAGCAAATATTAAATC
AAATATCCAAGGTAAGTGTACTATCACTCCGTTTTTACAT
GTTTGTTGGTTTGTATCTCTTCATAAATGGGGTAAATTTT
TCAAACCATTGAACTTCGAATGGCTTACGGATATTTTTAT
CCCCGCAGATTGCCGCTCACAACTACCAGATGATGATGAA
ATGAGACAGATGTACAGATATGGCGCTAACGTTGGATTTA
TTGACTTCACCCCCTGGATAAGCGAATTTGACATGAATGA
TAACAAAGAAAATTTTTGGCCACTTATTGAGCACTACCAT
GAAGTAATTTCGGAAGCTTTAAGAAATAAAAAGCATCTCC
ATGGCTTAGGGTTCAATATACAAGGCTTCGTTCAAAAATA
TGTGAACATTGACAAGGTAATGTGCGATCGTGCCATCGGG
AAAAGACGCGGAGGTACATTGTTAAGCAATGTAGGTCTGT
TTAATCAGTTAGAGGAGCCCGATGCCAAATATTCTATATG
CGATTTGGCATTTGGCCAATTTCAAGGATCCTGGCACCAA
GCATTTTCCTTGGGTGTTTGTTCGACTAATGTAAAGGGGA
TGAATATTGTTGTTGCTTCAACAAAGAATGTTGTTGGTAG
TCAAGAATCTCTCGAAGAGCTTTGCTCCATTTACAAAGCT
CTCCTTTTAGGCCCTTAG
Exemplary ATF1 protein
(UniProtKB-P40353)
(SEQ ID NO: 50)
(SEQ ID NO: 50)
MNEIDEKNQAPVQQECLKEMIQNGHARRMGSVEDLYVALN
RQNLYRNFCTYGELSDYCTRDQLTLALREICLKNPTLLHI
VLPTRWPNHENYYRSSEYYSRPHPVHDYISVLQELKLSGV
VLNEQPEYSAVMKQILEEFKNSKGSYTAKIFKLTTTLTIP
YFGPTGPSWRLICLPEEHTEKWKKFIFVSNHCMSDGRSSI
HFFHDLRDELNNIKTPPKKLDYIFKYEEDYQLLRKLPEPI
EKVIDFRPPYLFIPKSLLSGFIYNHLRFSSKGVCMRMDDV
EKTDDVVTEIINISPTEFQAIKANIKSNIQGKCTITPFLH
VCWFVSLHKWGKFFKPLNFEWLTDIFIPADCRSQLPDDDE
MRQMYRYGANVGFIDFTPWISEFDMNDNKENFWPLIEHYH
EVISEALRNKKHLHGLGFNIQGFVQKYVNIDKVMCDRAIG
KRRGGTLLSNVGLFNQLEEPDAKYSICDLAFGQFQGSWHQ
AFSLGVCSTNVKGMNIVVASTKNVVGSQESLEELCSIYKA
LLLGP
Exemplary ATF2 gene/mRNA
(GenBank Accession
No.: NM_001181306.1)
(SEQ ID NO: 51)
(SEQ ID NO: 51)
ATGGAAGATATAGAAGGATACGAACCACATATCACTCAAG
AGTTGATAGACCGTGGCCATGCAAGACGTATGGGCCACTT
GGAAAACTACTTTGCTGTTTTGAGTAGGCAGAAAATGTAC
TCGAATTTTACTGTTTACGCGGAATTGAATAAAGGTGTTA
ATAAGAGACAACTAATGCTTGTCTTGAAAGTATTACTTCA
AAAATACTCAACTCTTGCGCATACAATCATTCCTAAGCAT
TATCCTCATCATGAAGCGTACTACTCTAGCGAAGAGTACC
TTAGTAAACCTTTTCCACAGCATGATTTCATAAAGGTGAT
TTCTCATCTTGAATTCGATGACTTGATTATGAATAATCAA
CCAGAATACAGAGAAGTCATGGAGAAAATCTCAGAACAGT
TCAAAAAGGATGATTTCAAAGTCACCAATAGGTTAATCGA
ATTGATTAGCCCTGTAATCATACCTCTGGGTAATCCGAAG
AGGCCTAATTGGAGATTGATTTGTTTACCAGGTAAGGATA
CTGATGGGTTTGAAACGTGGAAAAACTTCGTTTATGTCAC
TAACCACTGCGGCTCCGACGGTGTCAGTGGATCGAATTTT
TTCAAAGATTTAGCTCTACTCTTTTGTAAAATCGAAGAAA
AAGGGTTTGATTATGATGAAGAGTTCATCGAAGATCAAGT
CATCATTGACTATGATCGAGACTACACTGAAATTTCTAAA
TTGCCAAAACCGATTACGGATCGTATTGACTACAAGCCAG
CATTGACTTCATTACCCAAATTCTTTTTAACAACCTTCAT
TTATGAACATTGTAATTTTAAAACCTCCAGCGAATCTACA
CTTACAGCTAGATATAGCCCCTCTAGTAATGCTAATGCTA
GTTACAATTACTTGTTGCATTTCAGTACTAAGCAAGTAGA
ACAAATCAGAGCTCAGATCAAGAAAAATGTTCACGATGGG
TGCACCCTAACACCCTTCATTCAAGCGTGCTTTCTTGTAG
CCCTGTATAGACTGGATAAGCTGTTCACAAAATCTCTTCT
CGAGTATGGGTTCGATGTGGCTATTCCAAGCAACGCAAGA
AGGTTTTTACCAAACGATGAAGAGTTAAGAGATTCTTATA
AATACGGCTCCAACGTTGGAGGTTCGCATTACGCCTATCT
AATCTCCTCATTCGACATTCCCGAAGGTGACAATGACAAG
TTTTGGAGTCTTGTCGAATACTACTATGACCGCTTTTTAG
AATCGTACGACAACGGTGACCACTTGATTGGTCTGGGGGT
CCTACAACTTGATTTTATCGTTGAAAACAAGAATATAGAC
AGCCTTCTTGCCAACTCTTATTTGCACCAGCAAAGAGGCG
GTGCAATCATCAGTAATACAGGACTTGTCTCGCAAGATAC
GACCAAGCCGTACTACGTTCGGGATTTAATCTTCTCGCAG
TCTGCAGGCGCCTTGAGATTTGCGTTCGGCCTAAACGTTT
GCTCCACAAACGTGAATGGTATGAACATGGACATGAGCGT
GGTTCAGGGCACTCTACGGGATCGTGGCGAATGGGAATCG
TTCTGCAAGCTCTTCTACCAAACCATCGGCGAATTTGCGT
CGCTTTAA
Exemplary ATF2 protein
(UniProtKB-P53296)
(SEQ ID NO: 52)
(SEQ ID NO: 52)
MEDIEGYEPHITQELIDRGHARRMGHLENYFAVLSRQKMY
SNFTVYAELNKGVNKRQLMLVLKVLLQKYSTLAHTIIPKH
YPHHEAYYSSEEYLSKPFPQHDFIKVISHLEFDDLIMNNQ
PEYREVMEKISEQFKKDDFKVTNRLIELISPVIIPLGNPK
RPNWRLICLPGKDTDGFETWKNFVYVTNHCGSDGVSGSNF
FKDLALLFCKIEEKGFDYDEEFIEDQVIIDYDRDYTEISK
LPKPITDRIDYKPALTSLPKFFLTTFIYEHCNFKTSSEST
LTARYSPSSNANASYNYLLHFSTKQVEQIRAQIKKNVHDG
CTLTPFIQACFLVALYRLDKLFTKSLLEYGFDVAIPSNAR
RFLPNDEELRDSYKYGSNVGGSHYAYLISSFDIPEGDNDK
FWSLVEYYYDRFLESYDNGDHLIGLGVLQLDFIVENKNID
SLLANSYLHQQRGGAIISNTGLVSQDTTKPYYVRDLIFSQ
SAGALRFAFGLNVCSTNVNGMNMDMSVVQGTLRDRGEWES
FCKLFYQTIGEFASL
Exemplary BAT2 gene or portion thereof
GenBank Accession No.:
NM_001181806.1
(SEQ ID NO: 53)
(SEQ ID NO: 53)
ATGACCTTGGCACCCCTAGACGCCTCCAAAGTTAAGATAA
CTACCACACAACATGCATCTAAGCCAAAACCGAACAGTGA
GTTAGTGTTTGGCAAGAGCTTCACGGACCACATGTTAACT
GCGGAATGGACAGCTGAAAAAGGGTGGGGTACCCCAGAGA
TTAAACCTTATCAAAATCTGTCTTTAGACCCTTCCGCGGT
GGTTTTCCATTATGCTTTTGAGCTATTCGAAGGGATGAAG
GCTTACAGAACGGTGGACAACAAAATTACAATGTTTCGTC
CAGATATGAATATGAAGCGCATGAATAAGTCTGCTCAGAG
AATCTGTTTGCCAACGTTCGACCCAGAAGAGTTGATTACC
CTAATTGGGAAACTGATCCAGCAAGATAAGTGCTTAGTTC
CTGAAGGAAAAGGTTACTCTTTATATATCAGGCCTACATT
AATCGGCACTACGGCCGGTTTAGGGGTTTCCACGCCTGAT
AGAGCCTTGCTATATGTCATTTGCTGCCCTGTGGGTCCTT
ATTACAAAACTGGATTTAAGGCGGTCAGACTGGAAGCCAC
TGATTATGCCACAAGAGCTTGGCCAGGAGGCTGTGGTGAC
AAGAAACTAGGTGCAAACTACGCCCCCTGCGTCCTGCCAC
AATTGCAAGCTGCTTCAAGGGGTTACCAACAAAATTTATG
GCTATTTGGTCCAAATAACAACATTACTGAAGTCGGCACC
ATGAATGCTTTTTTCGTGTTTAAAGATAGTAAAACGGGCA
AGAAGGAACTAGTTACTGCTCCACTAGACGGTACCATTTT
GGAAGGTGTTACTAGGGATTCCATTTTAAATCTTGCTAAA
GAAAGACTCGAACCAAGTGAATGGACCATTAGTGAACGCT
ACTTCACTATAGGCGAAGTTACTGAGAGATCCAAGAACGG
TGAACTACTTGAAGCCTTTGGTTCTGGTACTGCTGCGATT
GTTTCTCCCATTAAGGAAATCGGCTGGAAAGGCGAACAAA
TTAATATTCCGTTGTTGCCCGGCGAACAAACCGGTCCATT
GGCCAAAGAAGTTGCACAATGGATTAATGGAATCCAATAT
GGCGAGACTGAGCATGGCAATTGGTCAAGGGTTGTTACTG
ATTTGAACTGA
Exemplary BAT2 protein UniProt
ID P47176
(SEQ ID NO: 54)
(SEQ ID NO: 54)
MTLAPLDASKVKITTTQHASKPKPNSELVFGKSFTDHMLT
AEWTAEKGWGTPEIKPYQNLSLDPSAVVFHYAFELFEGMK
AYRTVDNKITMFRPDMNMKRMNKSAQRICLPTFDPEELIT
LIGKLIQQDKCLVPEGKGYSLYIRPTLIGTTAGLGVSTPD
RALLYVICCPVGPYYKTGFKAVRLEATDYATRAWPGGCGD
KKLGANYAPCVLPQLQAASRGYQQNLWLFGPNNNITEVGT
MNAFFVFKDSKTGKKELVTAPLDGTILEGVTRDSILNLAK
ERLEPSEWTISERYFTIGEVTERSKNGELLEAFGSGTAAI
VSPIKEIGWKGEQINIPLLPGEQTGPLAKEVAQWINGIQY
GETEHGNWSRVVTDLN
Exemplary THI3 gene or portion thereof-
SEQ ID NO: 55
(SEQ ID NO: 55)
ATGAATTCTAGCTATACACAGAGATATGCACTGCCGAAGT
GTATAGCAATATCAGATTATCTTTTCCATCGGCTCAACCA
GCTGAACATACATACCATATTTGGACTCTCCGGAGAATTT
AGCATGCCGTTGCTGGATAAACTATACAACATTCCGAACT
TACGATGGGCCGGTAATTCTAATGAGTTAAATGCTGCCTA
CGCAGCAGATGGATACTCACGACTAAAAGGCTTGGGATGT
CTCATAACAACCTTTGGTGTAGGCGAATTATCGGCAATCA
ATGGCGTGGCCGGATCTTACGCTGAACATGTAGGAATACT
TCACATAGTGGGTATGCCGCCAACAAGTGCACAAACGAAA
CAACTACTACTGCATCATACTCTGGGCAATGGTGATTTCA
CGGTATTTCATAGAATAGCCAGTGATGTAGCATGCTATAC
AACATTGATTATTGACTCTGAATTATGTGCCGACGAAGTC
GATAAGTGCATCAAAAAGGCTTGGATAGAACAGAGGCCAG
TATACATGGGCATGCCTGTCAACCAGGTAAATCTCCCGAT
TGAATCAGCAAGGCTTAATACACCTCTGGATTTACAATTG
CATAAAAACGACCCAGACGTAGAGAAAGAAGTTATTTCTC
GAATATTGAGTTTTATATACAAAAGCCAGAATCCGGCAAT
CATCGTAGATGCATGTACTAGTCGACAGAATTTAATCGAG
GAGACTAAAGAGCTTTGTAATAGGCTTAAATTTCCAGTTT
TTGTTACACCTATGGGTAAGGGTACAGTAAACGAAACAGA
CCCGCAATTTGGGGGCGTATTCACGGGCTCGATATCAGCC
CCAGAAGTAAGAGAAGTAGTTGATTTTGCCGATTTTATCA
TCGTCATTGGTTGCATGCTCTCCGAATTCAGCACGTCAAC
TTTCCACTTCCAATATAAAACTAAGAATTGTGCGCTACTA
TATTCTACATCTGTGAAATTGAAAAATGCCACATATCCTG
ACTTGAGCATTAAATTACTACTACAGAAAATATTAGCAAA
TCTTGATGAATCTAAACTGTCTTACCAACCAAGCGAACAA
CCCAGTATGATGGTTCCAAGACCTTACCCAGCAGGAAATG
TCCTCTTGAGACAAGAATGGGTCTGGAATGAAATATCCCA
TTGGTTCCAACCAGGTGACATAATCATAACAGAAACTGGT
GCTTCTGCATTTGGAGTTAACCAGACCAGATTTCCGGTAA
ATACACTAGGTATTTCGCAAGCTCTTTGGGGATCTGTCGG
ATATACAATGGGGGCGTGTCTTGGGGCAGAATTTGCTGTT
CAAGAGATAAACAAGGATAAATTCCCCGCAACTAAACATA
GAGTTATTCTGTTTATGGGTGACGGTGCTTTCCAATTGAC
AGTTCAAGAATTATCCACAATTGTTAAGTGGGGATTGACA
CCTTATATTTTTGTGATGAATAACCAAGGTTACTCTGTGG
ACAGGTTTTTGCATCACAGGTCAGATGCTAGTTATTACGA
TATCCAACCTTGGAACTACTTGGGATTATTGCGAGTATTT
GGTTGCACGAACTACGAAACGAAAAAAATTATTACTGTTG
GAGAATTCAGATCCATGATCAGTGACCCAAACTTTGCGAC
CAATGACAAAATTCGGATGATAGAGATTATGCTACCACCA
AGGGATGTTCCACAGGCTCTGCTTGACAGGTGGGTGGTAG
AAAAAGAACAGAGCAAACAAGTGCAAGAGGAGAACGAAAA
TTCTAGCGCAGTAAATACGCCAACTCCAGAATTCCAACCA
CTTCTAAAAAAAAATCAAGTTGGATACTGA
Exemplary THI3 protein-SEQ ID NO: 56
(SEQ ID NO: 56)
MNSSYTQRYALPKCIAISDYLFHRLNQLNIHTIFGLSGEF
SMPLLDKLYNIPNLRWAGNSNELNAAYAADGYSRLKGLGC
LITTFGVGELSAINGVAGSYAEHVGILHIVGMPPTSAQTK
QLLLHHTLGNGDFTVFHRIASDVACYTTLIIDSELCADEV
DKCIKKAWIEQRPVYMGMPVNQVNLPIESARLNTPLDLQL
HKNDPDVEKEVISRILSFIYKSQNPAIIVDACTSRQNLIE
ETKELCNRLKFPVFVTPMGKGTVNETDPQFGGVFTGSISA
PEVREVVDFADFIIVIGCMLSEFSTSTFHFQYKTKNCALL
YSTSVKLKNATYPDLSIKLLLQKILANLDESKLSYQPSEQ
PSMMVPRPYPAGNVLLRQEWVWNEISHWFQPGDIIITETG
ASAFGVNQTRFPVNTLGISQALWGSVGYTMGACLGAEFAV
QEINKDKFPATKHRVILFMGDGAFQLTVQELSTIVKWGLT
PYIFVMNNQGYSVDRFLHHRSDASYYDIQPWNYLGLLRVF
GCTNYETKKIITVGEFRSMISDPNFATNDKIRMIEIMLPP
RDVPQALLDRWVVEKEQSKQVQEENENSSAVNTPTPEFQP
LLKKNQVGY

Described in certain aspects is an engineered bacterium or population thereof, wherein the engineered bacterium having one or more polynucleotides that each encode one or more enzymes capable of catalyzing one or more reactions to produce a volatile compound, one or more polypeptides produced therefrom, or both, and wherein the engineered bacterium is of a strain that is a commensal bacterium strain found in the oral cavity of a mammal, optionally a canine or a feline.

In certain example embodiments, the one or more enzymes is/are
a. a methyltransferase that catalyzes the production of methyl salicylate from salicylic acid;
b. an isochorismate synthase;
c. an ischorisomate-pyruvate lyase;
d. is an alcohol acetyl transferase that is capable of condensing isoamyl alcohol and acetyl CoA into isoamyl acetate; or
e. any combination thereof.

In certain example embodiments, the one or more polynucleotides each encode a methyltransferase that catalyzes the production of methyl salicylate from salicylic acid, an isochorismate synthase, an ischorisomate-pyruvate lyase, or any combination thereof.

In certain example embodiments, the volatile compound produced has a mint odor.

In certain example embodiments, the one or more polynucleotides each comprise a PCHBA gene or mRNA, a BSMT1 gene or mRNA, or any combination thereof.

In certain example embodiments, at least one of the one or more polynucleotides encodes an alcohol acetyl transferase that is capable of condensing isoamyl alcohol and acetyl CoA into isoamyl acetate.

In certain example embodiments, the engineered bacterium or population thereof produces a fruity odor. In certain example embodiments, the fruity odor is a banana odor or a pear odor.

In certain example embodiments, at least one of the one or more polynucleotides includes an ATF gene or mRNA, an BAT2 gene or mRNA, a THI3 gene or mRNA, or any combination thereof.

In certain example embodiments, the ATF gene or mRNA is an ATF1 gene or mRNA or an ATF2 gene or mRNA.

In certain example embodiments, one or more of the one or more polynucleotides each comprise a gene or mRNA independently selected from a BSTM1 gene, a BSTM1 mRNA, an ATF gene, an ATF mRNA, a PCHBA gene, a PCHBA mRNA, a BAT2 gene, a BAT2 mRNA, a THI3 gene, a THI3 mRNA, or any combination thereof.

In certain example embodiments, the ATF gene or ATF mRNA is an ATF1 gene or mRNA or an ATF2 gene or mRNA.

In certain example embodiments,
a. the BSTM1 gene or mRNA has a polynucleotide sequence that is about 90-100 percent identical to any one of SEQ ID NO: 2, 8, 11, or 13;
b. the PCHBA gene or mRNA has a polynucleotide sequence that is about 90-100 percent identical to SEQ ID NO: 1 or 10;
c. the ATF gene or mRNA has a polynucleotide sequence that is about 90-100 percent identical to SEQ ID NO: 49 or 51;
d. the BAT2 gene or mRNA has a polynucleotide sequence that is about 90-100 percent identical to SEQ ID NO: 53;
e. the THI3 gene or mRNA has a polynucleotide sequence that is about 90-100 percent identical to SEQ ID NO: 55;
f. or any combination thereof.

In certain example embodiments, one or more of the one or more polynucleotides has a sequence that is about 40-100% identical to any one of SEQ ID NOs.: 1, 2, 8, 10-11, 13, 49, 51, 53, or 55, or any region therein of at least 20 nucleotides.

In certain example embodiments, one or more of the one or more polynucleotides encodes a polypeptide having a sequence that is about 40 to 100% identical to any one of SEQ ID NOs.: 3-7, 9, 50, 52, 54, or 56.

In certain example embodiments, the engineered bacterium or population thereof further contains one or more expression vectors, wherein the one or more expression vectors comprise the one or more polynucleotides.

In certain example embodiments, the engineered bacterium is from genus selected from Escherichia, Pseudomonas, Brevundimonas, Xanthomonas, Deinococcus, or Pedobacter.

In certain example embodiments, the engineered bacterium is an isolate of a commensal canine oral bacterium or population thereof.

In certain example embodiments, the commensal canine oral bacterium or population thereof is of the genus Pseudomonas.

In certain example embodiments, the engineered bacterium is an isolated commensal canine oral bacterium or population thereof having a genome that has one or more sequences that is/are 90-100 percent identical to any one or more of SEQ ID NOs: 12 or 14-48.

Described in certain aspects herein is an isolated commensal canine oral bacterium or population thereof having a genome having a sequence that is 90-100 percent identical to any one or more of SEQ ID NOs: 12 or 14-48.

In certain example embodiments, the isolated commensal canine oral bacterium or population thereof is of the genus Pseudomonas.

### Polynucleotides and Polypeptides Relevant to Producing Volatile Compounds

Described herein are polynucleotides relevant to producing a volatile compound in an engineered bacterium. Such polynucleotides can include one or more polypeptides relevant to and/or capable of producing, either alone or in combination with one or more other polypeptides, a volatile compound in a bacterium or other cell. In some embodiments, the engineered bacteria are engineered to contain and/or express or overexpress a polynucleotide corresponding to and/or containing a PCHBA, ATF1, ATF2, and/or BSMT1 gene. In some embodiments, the engineered bacteria are engineered to express and/or contain a PCHBA, ATF1, ATF2, and/or BSMT1 polypeptide. In some embodiments, the PCHBA, ATF1, ATF2, and/or BSMT1 gene and/or polypeptide is exogenous to the engineered bacteria. In some embodiments, the engineered bacteria express or overexpress a polynucleotide that encodes an alcohol acetyl transferase and/or alcohol acetyl transferase polypeptide that can condense isoamyl alcohol and acetyl CoA into isoamyl acetate. In some embodiments, the alcohol acetyl transferase encoding polynucleotide and/or polypeptide are exogenous to the engineered bacteria. In some embodiments, the engineered bacteria are engineered to contain and/or express or overexpress a polynucleotide that encodes a methyltransferase and/or a methyltransferase polypeptide that is involved in the biosynthesis of methyl salicylate from salicylic acid. In some embodiments, the methyltransferase encoding polynucleotide and/or polypeptide are exogenous to the engineered bacteria.

In some embodiments, the engineered bacteria are engineered to contain and/or express or overexpress a polynucleotide that is about 40 to 100 percent identical to any one of SEQ ID NOs.: 1, 2, 8, or 10-11 or any region of at least 20 consecutive nucleotides thereof. In some embodiments, the engineered bacteria are engineered to contain and/or express or overexpress a polynucleotide that is about 40 to/or 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent identical to any one of SEQ ID NOs.: 1, 2, 8, 10-11, 13, 49, 51, 53, or 55 or any region of at least 20 consecutive nucleotides thereof. In some embodiments, the engineered bacteria are engineered to contain and/or express or overexpress a polynucleotide that is about 95, to/or 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, or 100 percent identical to any one of SEQ ID NOs.: 1, 2, 8, 10-11, 13, 49, 51, 53, or 55 or any region of at least 20 consecutive nucleotides thereof.

In some embodiments, the engineered bacteria express or overexpress a methyl transferase that is involved in the biosynthesis of methyl salicylate from salicylic acid and is encoded by a BSMT1 polynucleotide. In some of these embodiments, the engineered bacteria are engineered to contain and/or express or overexpress a BSMT1 encoding polynucleotide. In some embodiments, the engineered bacteria can express or overexpress an alcohol acetyl transferase that can condense isoamyl alcohol and acetyl CoA into isoamyl acetate and is encoded by an ATF polynucleotide. In some of these embodiments, the engineered bacteria are engineered to contain and/or express or over express an ATF encoding polynucleotide. In some embodiments, the polynucleotide in the engineered bacteria encodes a polypeptide that is about 40 to 100 percent identical to any one of SEQ ID NOs.: 3-7, 9, 50, 52, 54, or 56 or any region of at least 20 consecutive nucleotides thereof. In some embodiments, the polynucleotide in the engineered bacteria encodes a polypeptide that is about 40 to/or 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent identical to any one of SEQ ID NOs.: 3-7, 9, 50, 52, 54, or 56 or any region of at least 20 consecutive nucleotides thereof. In some embodiments, the polynucleotide in the engineered bacteria encodes a polypeptide that is about 95, to/or 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, or 100 percent identical to any one of SEQ ID NOs.: 3-7, 9, 50, 52, 54, or 56 or any region of at least 20 consecutive nucleotides thereof.

In some embodiments, the bacteria are engineered to transiently express one or more polynucleotides and/or polypeptides described herein. In some embodiments, the bacteria are engineered to stably express one or more polynucleotides and/or polypeptides described herein.

In some embodiments the polynucleotide, vector, plasmid, and the like do not contain an antibiotic resistance selectable marker or an antibiotic resistance gene. In some embodiments, the engineered bacteria are engineered such that they do not contain exogenous or endogenous antibiotic resistance genes.

### Codon Optimization of Polynucleotides

The polynucleotide(s) relevant to producing a volatile compound in an engineered bacterium described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a polypeptide relevant to and/or capable of producing a volatile compound corresponds to the most frequently used codon for a particular amino acid. As to codon usage, a codon usage table can be referenced to determine bias and codon optimization of polynucleotides. Such tables for all species and tissues in GenBank and RefSeq can be found, for example, at the HIVE lab https://hive.biochemistry.gwu.edu. See also, e.g., Athey J., et al. A new and updated resource for codon usage tables. BMC Bionformatics, 2017; 18(1):391; Alexaki, A., et. al., C. Codon and Codon-Pair Usage Tables (CoCoPUTs): facilitating genetic variation analyses and recombinant gene design. Journal of Molecular Biology. 2019; and Kames, J., et al. TissueCoCoPUTs: novel human tissue-specific codon and codon-pair usage tables based on differential tissue gene expression. Journal of Molecular Biology. 2019; in press. Other resources for codon usage tables that can be appropriate for use with the present disclosure are available from GenScript at https://www.genscript.com/tools/codon-frequency-table, and include bacteria such as E. coli.

As to codon usage in bacteria, reference is made to the Microbial genome database available from NCBI at https://www.ncbi.nlm.nih.gov/genome/microbes/, the codon usage tables as previously discussed, and e.g., Ermolaeva., Curr. Issues Mol. Biol. (2001) 3(4): 91-97. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gown, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

In some embodiments, the polynucleotide(s) is/are codon optimized for expression in a prokaryote. In some embodiments, the polynucleotide(s) is/are codon optimized for expression in a bacterium. In some embodiments, the polynucleotide(s) is/are codon optimized for expression in bacteria of the genus Escherichia, Pseudomonas, Brevundimonas, Xanthomonas, Deinococcus, or Pedobacter. In some embodiments, the polynucleotide(s) is/are codon optimized for expression in E. coli.

In some embodiments, the BSTM1 polynucleotide, PCHBA polynucleotide, or ATF polynucleotide (ATF 1 or ATF2 polynucleotide) and/or other component of the expression vector can be codon optimized for expression in a specific bacterium strain or species. In some embodiments, the polynucleotide and/or expression BSTM1 polynucleotide, PCHBA polynucleotide, or ATF polynucleotide (ATF 1 or ATF2 polynucleotide) and/or other component of the expression vector can be codon optimized for expression in E. coli or a species of the genus Escherichia, Brevundimonas, Pseudomonas, Xanthomonas sp., Deinococcus sp., or Pedobacter sp.

### Vectors

Also described herein are vectors that can contain a polynucleotide described elsewhere herein, including but not limited to a PCHBA gene, (e.g., ATF1 and/or ATF2), a BAT2 gene, aTHI3 gene, and/or a BSMT1 gene. The vectors can be useful for producing the engineered bacteria described elsewhere herein. Other uses for the vectors and vector systems described herein are also within the scope of this disclosure. In some embodiments, the vector is an expression vector. In some embodiments, the vector is a plasmid. In some embodiments, the vector is a bacterial plasmid, derived from a bacterial plasmid, or contain one or more components from a bacterial plasmid. In some embodiments the vector does not contain an antibiotic resistance gene.

Described in certain aspects is an expression vector capable of expressing a BSTM1 polypeptide, a PCHB polypeptide, a PCHA polypeptide, an ATF1 polypeptide, an ATF2 polypeptide, BAT2 polypeptide, a THI3 polypeptide or any combination thereof, the expression vector containing:
a. a BSTM1 polynucleotide having a sequence that is about 90-100 percent identical to any one of SEQ ID NO: 2, 8, 11, or 13;
b. a PCHBA polynucleotide having a sequence that is about 90-100 percent identical to SEQ ID NO: 1 or 10;
c. an ATF polynucleotide having a sequence that is about 90-100 percent identical to SEQ ID NO: 49 or 51;
d. a BAT2 polynucleotide having a sequence that is about 90-100 percent identical to SEQ ID NO: 53;
e. a THI3 polynucleotide having a sequence that is about 90-100 percent identical to SEQ ID NO: 55;
f. or any combination thereof.

In certain example embodiments, (a), (b), (c), (d), (e), or (f) of the expression vector is/are operatively coupled one or more regulatory elements.

In certain example embodiments, the expression vector contains a sequence that is 90-100 percent identical to SEQ ID NO: 13.

In some embodiments, the vector is capable of autonomous self-replication in a host cell into which it is introduced. In some embodiments, the vector is not capable of self-replication in a host cell into which it is introduced.

Vectors include, but are not limited to, nucleic acid molecules that are single-stranded, double-stranded, or partially double-stranded; nucleic acid molecules that comprise one or more free ends, no free ends (e.g., circular); nucleic acid molecules that contain DNA, RNA, or both; and other varieties of polynucleotides known in the art.

In some embodiments, the vector facilitates integration, such as stable integration, of one or more of its polynucleotides into a host's genome. Such polynucleotides are replicated along with the host's cell genome. In some embodiment, the vector is configured for transient expression of one or more polynucleotides contained therein.

In some embodiments, the vector is capable of directing the expression of one or more polynucleotides to which they are operatively linked. Such vectors are also known as the term of art “expression vectors”. Expression vectors can be composed of a nucleic acid (e.g., a polynucleotide) of the invention in a form suitable for expression of the nucleic acid in a host cell, which means that the expression vectors include one or more regulatory elements, which can be selected on the basis of the host cells to be used for expression, that is operatively-linked to the nucleic acid sequence to be expressed. Within an expression vector, “operably linked” and “operatively-linked” are used interchangeably herein and further defined elsewhere herein.

In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some embodiments the vector can be a bicistronic vector. In some embodiments, the bicistronic vector contains and/or is capable of expressing one or more of the polynucleotides described herein, including, but not limited to, a PCHBA gene, (e.g., ATF1 and/or ATF2), a BAT2 gene, aTHI3 gene, and/or a BSMT1 gene.

The vectors can be viral-based or non-viral based.

In some embodiments, the vector is a bacterial expression vector. Bacterial expression vectors are generally known in the art. Any suitable bacterial expression vector or component thereof can be used in the context of the present disclosure, Exemplary, non-limiting bacterial expression vectors are the pGEM series of vectors (available from Promega), pGEX series of vectors, the pET series of vectors (see e.g., Bolivar, F., et al. (1977). Gene 2, 95-113. doi: 10.1016/0378-1119(77) 90000-2, Novagen), the pUC series of vectors (see e.g., Minton, N. P. (1984). Gene 31, 269-273. doi: 10.1016/0378-1119(84)90220-8) (e.g., pUC18, pUC19, etc., the pQE series of vectors (available from Qiagen), the Gateway Cloning series of vectors, a dual plasmid expression vector system (e.g., the pACYC series of vectors, the pBAD series of vectors, see e.g., Chang, A. C., and Cohen, S. N. (1978). J. Bacteriol. 134, 1141-1156 and Guzman et al. (1995). J. Bacteriol. 177, 4121-4130), a triple plasmid expression vector system (e.g., the pSC101 plasmid or variant thereof, see e.g., Nordstrom, K. (2006). Plasmid 55, 1-26.doi: 10.1016/j.plasmid.2005.07.002), the pBR series of vectors (e.g., pBR322, pBR322, pBR327, etc.), the pME series of vectors (see e.g., pME6010), the pBBR series of vectors (e.g., pBBR1, see also e.g., Ouahrani-Bettache et al. BioTechniques 26:620-622 (April 1999).

In some embodiments, the vector can be a yeast expression vector or can contain an element or construct thereof. Examples of yeast expression vectors include, but are not limited to, pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif), and those set forth in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of yeast expression vectors include plasmids, yeast artificial chromosomes, 2p, plasmids, yeast integrative plasmids, yeast replicative plasmids, shuttle vectors, and episomal plasmids. In some embodiments, the yeast expression vector is capable of expression of one or more polynucleotides in a bacterium.

In some embodiments, the vector is a baculovirus vector or expression vector can contain an element or construct thereof. Baculovirus expression vectors are generally known in the art and include, without limitation, the pAc series (Smith, et al., 1983. Mol. Cell. Biol. 3: 2156-2165) and the pVL series (Lucklow and Summers, 1989. Virology 170: 31-39). In some embodiments, the baculovirus expression vector is capable of expression of one or more polynucleotides in a bacterium.

In some embodiments, the vector is a mammalian expression vector or can contain an element or construct thereof. Mammalian expression vectors are generally known in the art Examples of mammalian expression vectors include, but are not limited to, a pSV vector, a pCMV vector, pCDM8 (Seed, 1987. Nature 329: 840) and pMT2PC (Kaufman, et al., 1987. EMBO J. 6: 187-195). In some embodiments, the mammalian expression vector is capable of expression of one or more polynucleotides in a bacterium.

In some embodiments, the vector is a plant expression vector or can contain an element or construct thereof. Plant expression vectors are generally known in the art. In some embodiments, the plant expression vector is capable of expression of one or more polynucleotides in a bacterium.

In some embodiments, the BSTM1 polynucleotide, PCHBA polynucleotide, and/or ATF polynucleotide (ATF 1 or ATF2 polynucleotide) is included in a vector, such as a suitable expression vector or cloning vector. In some embodiments, the BSTM1 polynucleotide, PCHBA polynucleotide, or ATF polynucleotide (ATF 1 or ATF2 polynucleotide) is included in a plasmid or artificial chromosome.

In some embodiments the vector is a Gateway cloning vector (e.g., pDONR207). In some embodiments, the vector is a destination vector (e.g., MTN41 (for E. coli, plus a few other bacteria species) and pBAV226 (different origin of replication then MTN41), which both have antibiotic resistance markers). Other suitable vectors include, but are not limited to, plasmid derivatives of pBBR1 and pME6010 plasmids as well as a Tn7 transposon. In certain example embodiments, the expression vector capable of expressing a BSTM1 enzyme can have a polynucleotide having a sequence that is about 90-100% identical to SEQ ID NO: 13. It will be appreciated that the engineered bacteria can be engineered to contain expression vector capable of expressing a BSTM1 enzyme can have a polynucleotide having a sequence that is about 90-100% identical to SEQ ID NO: 13.

Any of the cDNA, DNA, or other polynucleotide sequences described herein can be incorporated into a suitable expression vector. The expression vector can contain one or more regulatory sequences, or one or more other sequences used to facilitate the expression of an odor polynucleotide previously described. The expression vector can contain one or more regulatory sequences and/or one or more other sequences used to facilitate the replication of the odor producing expression vector. The expression vector can be suitable for expressing a methyl transferase that is involved in the biosynthesis of methyl salicylate from salicylic acid and is encoded by a BSMT1 polynucleotide in a bacterial cell. The expression vector can be suitable for expressing express an alcohol acetyl transferase that can condense isoamyl alcohol and acetyl CoA into isoamyl acetate and is encoded by an ATF polynucleotide.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

Additional suitable cloning, expression and other vectors will be appreciated by those of ordinary skill in the art.

### Vector Features

The vector can contain one or more additional features, elements, and/or constructs in addition to the one or more polynucleotides that encode a gene relevant to producing a volatile compound in an engineered bacteria as is described elsewhere herein. The additional features that can confer one or more functionalities to the vector, the polynucleotide to be delivered, a virus particle produced there from, or polypeptide expressed thereof. Such features include, but are not limited to, promoters and/or regulatory elements, selectable markers, molecular identifiers (e.g., molecular barcodes), stabilizing elements, replicons, multiple cloning sites, and the like. It will be appreciated by those skilled in the art that the design of the expression vector and additional features included can depend on such factors as the choice of the host cell to be transformed, the level of expression desired, copy number desired, integration type (stable or transient), etc.

### Replicons

Replicons are elements that facilitate replication of genetic elements to which they are associated with as autonomous units. Replicons are generally known in the art and can contain one origin of replication together with its associated cis-acting control elements. Replicons control the copy number. See e.g., del Solar, G., and Espinosa, M. (2000). Plasmid copy number control: an ever-growing story. Mol. Microbiol. 37, 492-500. doi: 10.1046/j.1365-2958.2000.02005.x. In some embodiments, the replicon is a low (less than 20 copies per cell) or medium copy (20-100 copies per cell) number replicon. Without being bound by theory, can facilitate management of the metabolic stress producing an exogenous protein can put on the host cell. In some embodiments, the replicon is a high copy number replicon (more than 100 copies per cell). In some embodiments, the vector is a low copy number vector. In some embodiments, the vector is a medium copy number vector. In some embodiments, the vector is a high copy number vector.

In some embodiments, a replicon included in the vector facilitates production of less than 20 copies per cell. some embodiments, a replicon included in the vector facilitates production of 20 to 100 copies per cell. some embodiments, a replicon included in the vector facilitates production of more than 100 copies per cell. some embodiments, a replicon included in the vector facilitates production of about 500 to 700 copies per cell. In some embodiments, the replicon facilitates production of about 10 to 1,000, 10-20, 10-30, 10-40, 10-50, 10-75, 10-100, 10-200, 10-300, 10-400, 10-500, 500-100, 200-500, 300-500, 400-500, 500-600, 500-700, 500-800, or 500-900 copies of the vector or more per cell. In some embodiments the replicon In some embodiments, the replicon produces 10, to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 or more or any range of values therein.

### Regulatory Elements

The vector can include one or more regulatory elements, which can optionally be coupled to a polynucleotide that encode a gene relevant to producing a volatile compound in an engineered bacteria as is described elsewhere herein.

Regulatory elements include those that direct constitutive expression of a nucleotide sequence in many types of host cell and those that direct expression of the nucleotide sequence only in certain host cells (e.g., species specific, tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type or species specific.

To express a polynucleotide, the vector can include one or more transcriptional and/or translational initiation regulatory sequences, e.g., promoters, that direct the transcription of the gene and/or translation of the encoded protein in a cell. In some embodiments, the promoter is a constitutive promoter. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter or lac operon for bacterial expression and an alcohol dehydrogenase promoter for expression in yeast.

In some embodiments, a vector includes one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. In some embodiments, the vector includes one or more enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory element can be a regulated promoter. “Regulated promoter” refers to promoters that direct gene expression not constitutively, but in a temporally- and/or spatially regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development.

In some embodiments, the regulated promoter is an inducible/conditional promoter. Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which, are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector can contain a minimal promoter.

In some exemplary embodiments, a BSTM1 polynucleotide, PCHBA polynucleotide, and/or ATF polynucleotide (ATF 1 or ATF2 polynucleotide) are each operatively coupled to a regulatory polynucleotide including, but not limited to, a promoter. In some embodiments, the promoter causes expression of a polynucleotide in a bacterial cell. In some embodiments, the promoter can be a MPT2 promoter.

### Translocation Elements

In some embodiments, the vector and/or polynucleotide includes one or more elements capable of translocating and/or expressing a polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

The vector can include one or more selectable markers or tags. In some embodiments, one or more of the polynucleotides relevant to producing a volatile compound is/are operably linked, fused to, or otherwise modified to include a polynucleotide that encodes or is a selectable marker or tag, which can be a polynucleotide or polypeptide. In some embodiments, the polypeptide encoding a polypeptide selectable marker or tag can be incorporated in a polynucleotide relevant to producing a volatile compound such that the selectable marker or tag polypeptide, when translated, is inserted between two amino acids between the N- and C-terminus of the polypeptide relevant to producing a volatile compound or at the N- and/or C-terminus of the polypeptide relevant to producing a volatile compound. In some embodiments, the selectable marker or tag is a polynucleotide barcode or unique molecular identifier (UMI).

It will be appreciated that the polynucleotide encoding such selectable markers or tags can be incorporated into a polynucleotide encoding a polypeptide relevant to production of a volatile compound described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more polypeptides relevant to producing a volatile compound described herein via suitable linker. Suitable linkers are generally known in the art and include, but are not limited to, glycine or glycine serine linkers as short as GS or GG up to (GGGGG)3 (SEQ ID NO: 57) or (GGGGS)3(SEQ ID NO: 58). Such linkers can be encoded by the polynucleotides relevant to producing a volatile compound described herein.

### Codon Optimization of Vector Polynucleotides

As described elsewhere herein, the polynucleotide(s) relevant to producing a volatile compound in an engineered bacterium described herein can be codon optimized. In some embodiments, one or more polynucleotides contained in a vector (“vector polynucleotides”) described herein that are in addition to an optionally codon optimized polynucleotide encoding aspects of the relevant to producing a volatile compound in an engineered bacterium described herein can be codon optimized. The general principles of codon optimization are discussed elsewhere herein. In some embodiments, the vector polynucleotide is codon optimized for expression in a prokaryote. In some embodiments, the vector polynucleotide is codon optimized for expression in a bacteria or yeast. In some embodiments, the vector polynucleotide(s) is/are codon optimized for expression in bacteria of the genus Escherichia, Pseudomonas, Brevundimonas, Xanthomonas, Deinococcus, or Pedobacter. In some embodiments, the vector polynucleotide(s) is/are codon optimized for expression in E. coli.

### Engineered Bacteria Species

The engineered bacteria containing and/or expressing a polynucleotide and/or polypeptide relevant to producing a volatile compound described herein can be any suitable species, strain, or variant. In other words, a host bacterium that is engineered with one or more genes such that they produce a volatile compound as described herein can be any suitable species, strain, or variant. In some embodiments, the engineered bacteria species is an isolate strain described elsewhere herein. In some embodiments, the engineered bacteria species is a canine or feline commensal bacteria. In some embodiments, the engineered bacteria species is native to a feline or canine oral commensal microflora. In some embodiments, the engineered bacteria species is non-pathogenic. In some embodiments, the engineered bacteria species is not resistant to antibiotics.

In some embodiments, the engineered bacteria are of the genus Escherichia. In some embodiments, the engineered bacteria are E. coli. In some embodiments, the engineered bacteria are of the genus Pseudomonas. In embodiments, the engineered bacteria are non-pathogenic. In some embodiments, the engineered bacteria are of the genus Brevundimonas. In some embodiments, the engineered bacteria are of the genus Xanthomonas. In some embodiments, the engineered bacteria are of the genus Deinococcus. In some embodiments, the engineered bacteria are of the genus Pedobacter.

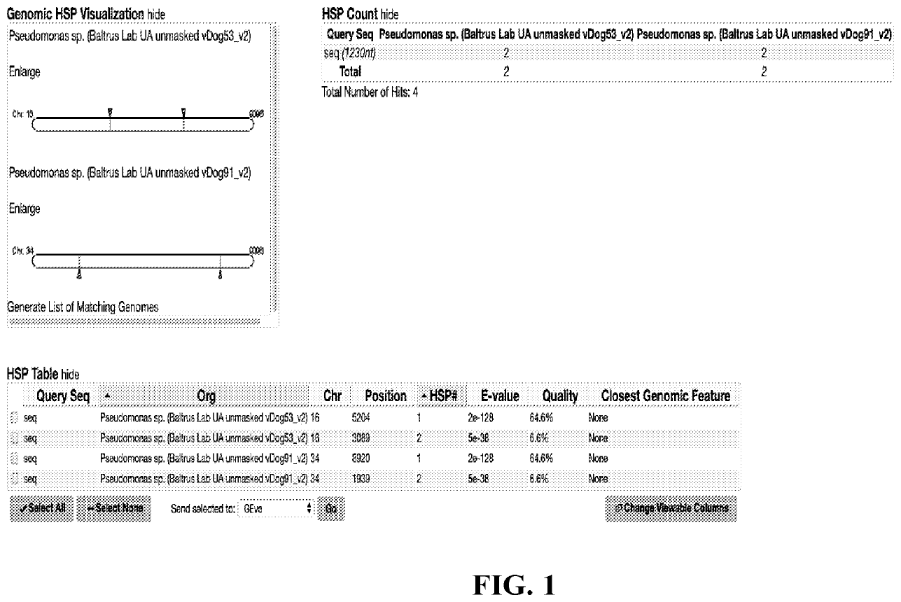

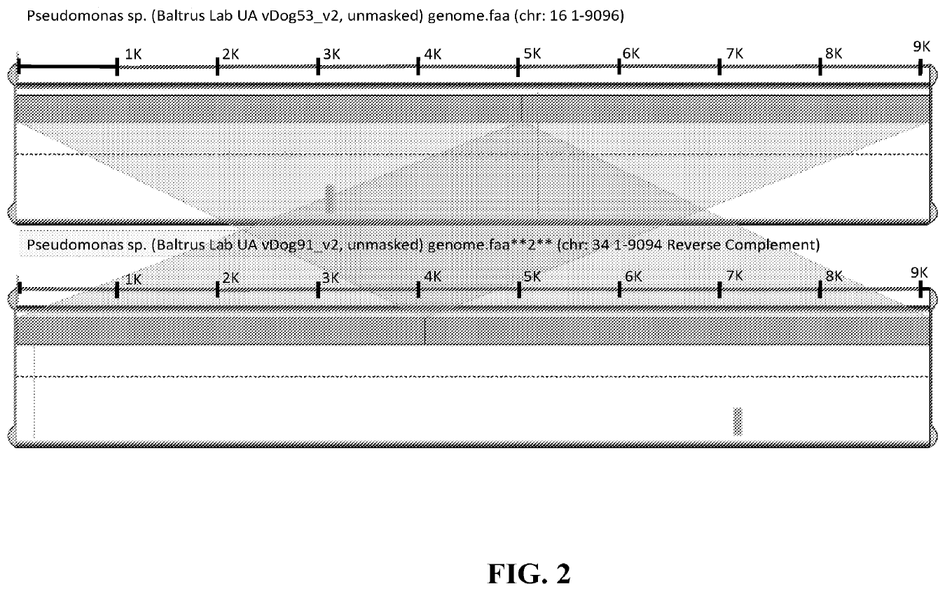

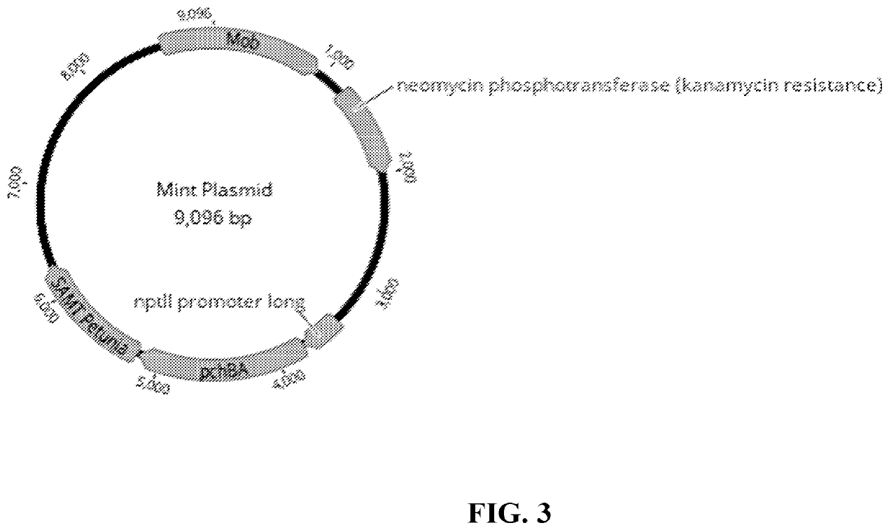

In some embodiments, the engineered bacteria are part of the canine oral microbiome. In some embodiments, the engineered bacteria are any one as set forth in Dewhirst et al. PloS One. 2012. 7(4): e36067, particularly at FIGS. 1-4 and Tables S1, S2, S3 and S4, such as one having any one of Genbank Accession number JN713151-JN713566. In some embodiments, the engineered bacteria are any one as set forth in Bell et al., 2020. Front. Vet Sci. https://doi.org/10.3389/fvets0.2020.00616, particularly at Table 3 and Supplementary Table 1. In some embodiments, the engineered bacteria are any one as set forth in Ruparell et al., 2020. BMC Microbiology. 20:42, particularly at FIG. 2, FIG. 3, and Table 1. In some embodiments, the engineered bacteria are any one as set forth in Sturgeon et al., 2013. 162:891-898. In some embodiments, the engineered bacteria are any one as set forth in Oh et al. 2015. PloS One. 10(7):e0131468, particularly at FIG. 2, FIG. 3, Table 3, and Table 4.

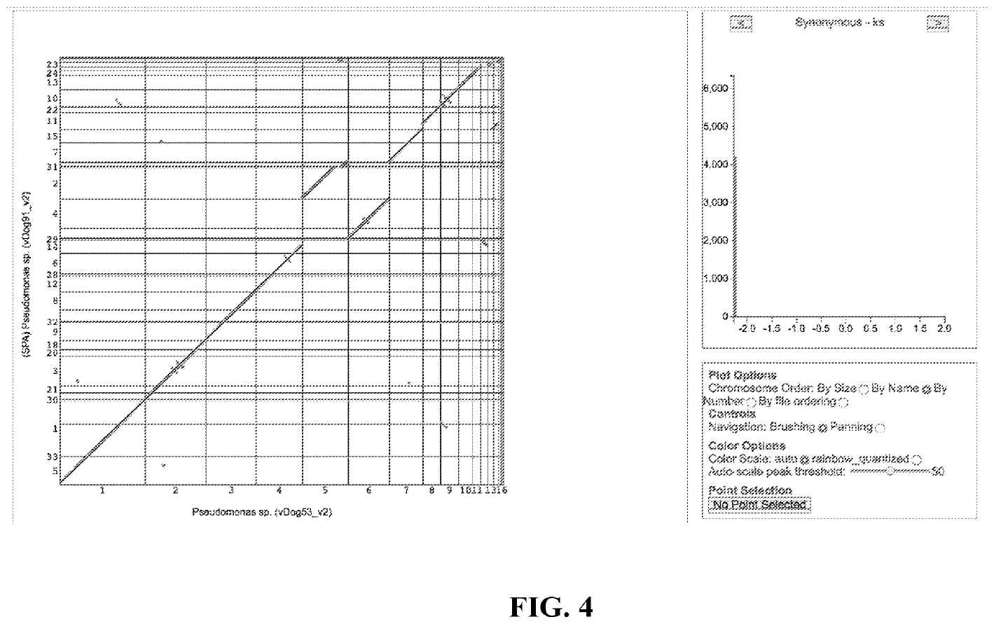

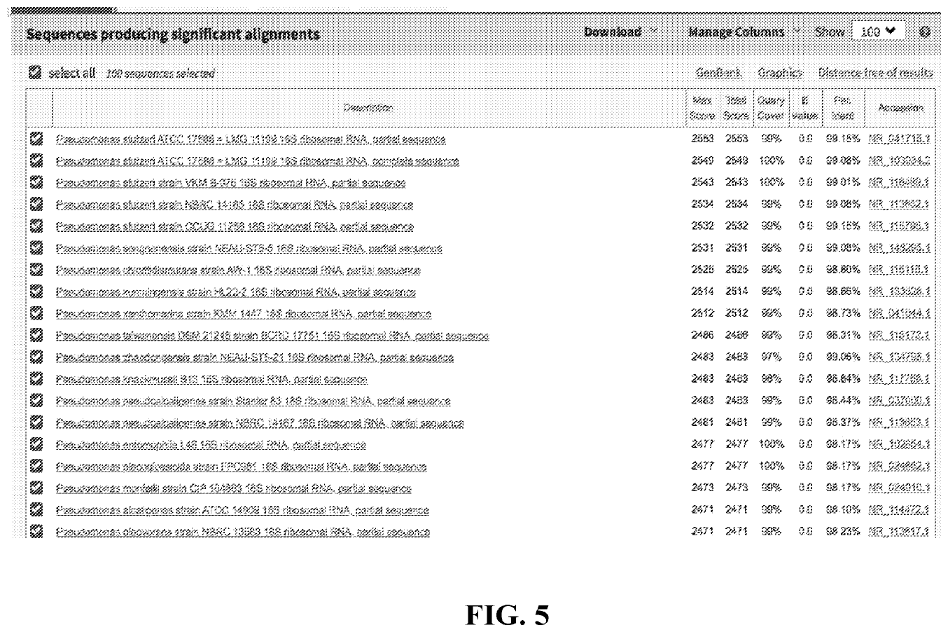

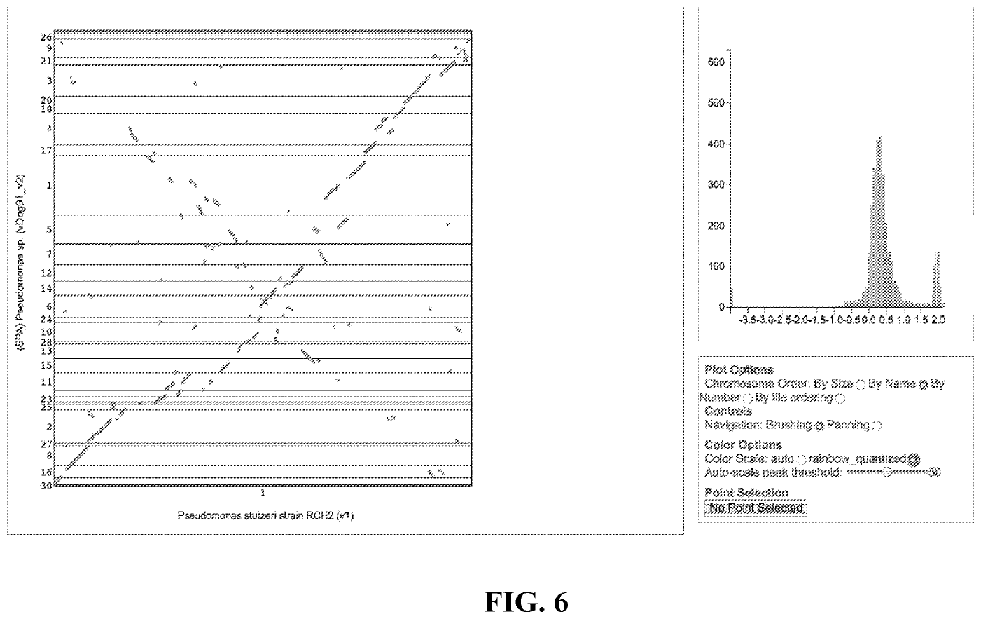

In some embodiments, the engineered bacteria are part of the feline oral microbiome. In some embodiments, the engineered bacteria are any one as set forth in Krumbeck et al., 2021. Pathogens. 10(904): https://doi.org/10.3390/pathogens10070904, particularly at FIG. 3, FIG. 4, FIG. 5, FIG. 6, FIG. S1, Table S1. In some embodiments, the engineered bacteria are any one as set forth in Older et al. 2019, particularly at FIG. 1, FIG. 3, FIG. 4, FIG. 5, FIG. S1, FIG. S2, FIG. S3, FIG. S4, FIG. S5, Table 2, Table S3, and Table S5.

### Isolated Strains

Also described in certain example embodiments herein are isolated strains of commensal oral bacteria. In some embodiments, the isolated strain of commensal oral bacteria are suitable for engineering to produce one or more volatile compounds as described elsewhere herein. In some embodiments, the isolate strain of commensal canine oral bacterium can be of the “Dog 53”, “Dog 91” or “Dog95” isolate or a variant thereof as demonstrated at least in the Working Examples herein.

In certain example embodiments, an isolated strain of commensal canine oral bacterium or population thereof suitable for engineering as described elsewhere herein can have a genome containing a sequence as in any one or more of SEQ ID NOs: 12 or 14-48. In certain example embodiments, the isolated commensal canine oral bacterium or population thereof is of the genus Pseudomonas. Further details of such strains are in the Working Examples below.

In some embodiments, the bacteria to be engineered to express a polynucleotide and/or polypeptide relevant to producing a volatile compound can be identified by the following procedure and selection criteria. Commensal oral bacteria of dogs or cats that are suitable for expression of an exogenous gene as described herein can be isolated and typed to determine strains isolated. Strains that meet the following selection criteria can be engineered to transiently or stably express an odor producing gene as described above. Isolated strains can be, for example, streaked on agar plates and grown in a suitable media (e.g., R2A) media. Suitable bacteria can be fast growing when grown on standard medium plates. As used in this context “fast growing” refers to colonies that appear within 2-7 days on an R2A agar plate Fer streaking on the R2A (or other standard media) plate. Fast growing and non-pathogenic strains can be characterized and identified using rRNA genotyping. Strains with natural antibiotic resistance can be differentiated from strains that do not have a natural antibiotic resistance. In some embodiments, strains suitable for engineering as described herein will not have a natural antibiotic resistance as identification of the engineered strains can be identified by testing for an antibiotic resistance selectable marker via exposing the engineered cells to an antibiotic. If the bacteria are naturally antibiotic resistant, the antibiotic resistance selectable marker will not be able to function as intended. In some of these embodiments, the antibiotic marker can be tailored so as to be different than the natural resistance. Further, it is desirable for the bacteria strain to have a codon bias (genomic/coding GC content) that is similar to E. coli. This can be used to objectively identify target commensal oral bacterial strains that will be amenable to expressing the odor producing constructs that are validated in E. coli.

### Methods of Engineering Bacteria

Methods of transforming or transducing, both stably and transiently, bacterial cells are generally known in the art. Methods of cloning bacterial cells are generally known in the art. Methods of screening for transformed/transduced bacterial cells are dependent on the selectable marker used and will be appreciated by one of ordinary skill in the art.

### Engineered Volatile-Producing Bacteria Formulations

Also described herein are formulations that can contain an amount, effective amount, and/or least effective amount, and/or therapeutically effective amount of one or more compounds, molecules, compositions, vectors, vector systems, cells (e.g., engineered bacterial cells described herein), or a combination thereof (which are also referred to as the primary active agent or ingredient elsewhere herein) described in greater detail elsewhere herein a pharmaceutically acceptable carrier. In some embodiments, the formulation can be a liquid adapted for oral administration. In some embodiments, the formulation can be a solid, semi-solid, paste, gel, and the like that can be filled into a void on an object (described elsewhere herein). In some embodiments, the formulation can be suitable as a coating that can be applied to an object for delivery of the engineered bacterial cells. The engineered bacterial cells can be incorporated into a food item or treat that can be masticated and ingested. Generally, the formulation can be administered orally to the animal. The engineered bacterial cells can be incorporated into a solution, semi-solid, or other solid that can be otherwise administered to the oral cavity of a subject. In some embodiments, the solution, semi-solid, or other solid can be biodegradable. In some embodiments, the subject is a mammal. In some embodiments, the subject is a dog. In some embodiments, the dog is a cat.

Described in certain aspects is a formulation having an engineered bacterium as described herein, an expression vector of as described herein, or both, optionally wherein the engineered bacterium includes the expression vector and a liquid, semi-solid, or solid carrier.

In certain example embodiments, the formulation is a foodstuff suitable for a mammal. In certain example embodiments, the mammal is a non-human animal. In certain example embodiments, the mammal is a canine or a feline.

In certain example embodiments, the formulation is a liquid solution or semi-solid suitable for administration to the oral cavity of a mammal. In certain example embodiments, the mammal is a non-human animal. In certain example embodiments, the mammal is a canine or a feline.

In certain example embodiments, the formulation is a formed object that is configured to be chewed on by a mammal. In certain example embodiments, the mammal is a non-human animal. In certain example embodiments, the mammal is a canine or a feline.

### Pharmaceutically Acceptable Carriers and Auxiliary Ingredients and Agents

The formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of auxiliary active agents, including anti-infectives, such as antimicrobials. In some embodiments, the anti-infective is an anti-infective compound in which the engineered bacteria is resistant to such that the inclusion of the anti-infective compound does not affect the engineered bacteria's ability to produce volatile compounds.

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

### Effective Amounts

In some embodiments, the amount of the primary active agent (e.g., engineered bacteria) and/or optional auxiliary active agent can be an effective amount, least effective amount, and/or therapeutically effective amount. The effective amount, least effective amount, and/or therapeutically effective amount of the primary and/or optional auxiliary active agent described elsewhere herein when present in the formulation can range from any non-zero amount about 0 to 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges. In some embodiments, the effective amount, least effective amount, and/or therapeutically effective amount of the primary and/or optional auxiliary active agent described elsewhere herein can be an effective concentration, least effective concentration, and/or therapeutically effective concentration, which each can be, when present in the formulation, any non-zero value ranging from about 0 to 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, mM, or M or be any numerical value with any of these ranges.

In other embodiments, the effective amount, least effective amount, and/or therapeutically effective amount of the auxiliary active agent, when present in the formulation, can be any non-zero value ranging from about 0.001, to 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, a primary active agent can be present in the pharmaceutical formulation at any non-zero value ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the formulation.

In some embodiments, the auxiliary active agent, when optionally present, can be included at any non-zero value ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the formulation.

In some embodiments, where a cell population is delivered, the effective amount of cells can range from about 1×101/mL or μL to 1×1020/mL or μL or more, such as about 1×101/mL or μL, 1×102/mL or μL, 1×103/mL or μL, 1×104/mL or μL, 1×105/mL or μL, 1×106/mL or μL, 1×107/mL or μL, 1×108/mL or μL, 1×109/mL or μL, 1×1010/mL or μL, 1×1011/mL or μL, 1×1012/mL or μL, 1×1013/mL or μL, 1×1014/mL or μL, 1×1015/mL or μL, 1×1016/mL or μL, 1×1017/mL or μL, 1×1018/mL or μL, 1×1019/mL or μL, to/or about 1×1020/mL or μL or more.

In some embodiments, the cell population included in the formulation contains about 1; 10; 100; 1000; 10,000; 100,000; 1-100 million, 0.5 billion, 1 billion, 2 billion, 3 billion, 4 billion, 5 billion, 6 billion, 7 billion, 8 billion, 9 billion, 10 billion, 11 billion, 12 billion, 13 billion, 14 billion, 15 billion, 16 billion, 17 billion, 18 billion, 19 billion, 20 billion, 25 billion, 30 billion, 35 billion, 40 billion, 45 billion, 50 billion, or more colony forming units (CFUs) per dose, per mL, per μL, per μg, per mg, or per gram.

In embodiments where an auxiliary active agent is present in the formulation, the effective amount of the auxiliary active agent will vary depending on the auxiliary active agent.

When optionally present in the formulation, the auxiliary active agent can be included in the formulation or can exist as a stand-alone compound or formulation that can be administered contemporaneously or sequentially with the compound, derivative thereof, or formulation thereof. In yet other embodiments, the effective amount of the auxiliary active agent, when present, can be any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total auxiliary active agent in the formulation. In additional embodiments, the effective amount of the auxiliary active agent, when present, can be any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total formulation.

### Dosage Forms

In some embodiments, the formulations described herein can be in a dosage form. The dosage form can be administered to a subject in need thereof. The dosage form can be effective generate specific concentration or amount, such as an effective concentration or amount, at a given site in the subject in need thereof. In some cases, the dosage form contains a greater amount of the active ingredient than the final intended amount needed to reach a specific region or location within the subject, such as the mouth.

The dosage forms can be adapted for oral administration. Dosage forms adapted for oral administration can discrete dosage units such as capsules, pellets or tablets, powders or granules, solutions, or suspensions in aqueous or non-aqueous liquids; edible foams or whips, gels, pastes, powders, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be encapsulated or microencapsulated. The ingredients, such as inactive ingredients and optional auxiliary active agents, when included, can be generally recognized as safe (GRAS) ingredients and auxiliary active agents.

In some embodiments, the formulation is provided as a concentrate that is formulated to be diluted in a suitable diluent to a dosage form for use. The concentrate can be a liquid, semisolid, or solid. In some embodiments, the diluent is a liquid or semisolid. In some embodiments, the concentrate is diluted 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 fold or more to produce the dosage for use.

In some embodiments, the formulation is a solid form formulated to be incorporated into an animal food, such as incorporated within or applied to the surface of an animal food or treat such as a kibble, dehydrated, wet food (e.g., loaf, chunks in gravy etc.), dry animal treats (such as baked biscuits), soft animal treats, etc. Incorporation can occur at the site of manufacture or at the point of use.

In some embodiments, the dosage form is formulated for incorporation into a water source. In some embodiments, the dosage form is a solid form that can be dissolved in a suitable amount of water. In some embodiments, the dosage form is a liquid or semisolid form that can be dissolved in a suitable amount of water. In some embodiments, the liquid form is provided in a dissolvable capsule.

In some embodiments, the dosage form is a solid, such as a tablet or powder, formulated to be reconstituted in a liquid or semisolid to a dosage form for use.

In some embodiments, the dosage form is a gel or paste formulated to be applied to the oral cavity of an animal, such as a canine or feline. In some embodiments, the gel or paste can be applied by hand or with a device such as a toothbrush, such as an animal toothbrush.

In some embodiments, the dosage form is formulated to be applied to or in a toy such as at the point of manufacture or use, such that the dosage form is delivered to the oral cavity of the animal when the animal chews on, licks, or otherwise interacts with the toy with its mouth.

For some embodiments, the dosage form contains a predetermined amount of a primary active agent, auxiliary active ingredient, or both where appropriate per unit dose. In other embodiments, the predetermined amount of a primary active agent, auxiliary active agent, or both where appropriate, can be an appropriate fraction of the effective amount of the active ingredient. Such unit doses may therefore be administered once or more than once a day, month, or year (e.g., 1, 2, 3, 4, 5, 6, or more times per day, month, or year). Such formulations may be prepared by any of the methods well known in the art.

### Objects Containing the Engineered Volatile-Producing Bacteria

The engineered bacterial cells can be incorporated into an article that would be chewed on or licked by a dog or a cat (such as a toy or chew) but not necessarily ingested. Suitable toys can be bones, balls, and other formed objects. The formed objects can be any suitable three-dimensional shape and sized. The objects can be shaped and sized for accommodating different sized animals. The objects can be made from any suitable material, which are generally known in the art. The suitable material can be synthetic, natural, or a combination thereof. The objects can be coated with the engineered bacteria or a formulation thereof. The objects can include one or more voids, pockets, grooves, or other region shaped to hold a liquid, gel, paste, or solid composition. In some embodiments, the objects can include a formulation containing the engineered bacteria in one of those voids pockets, grooves, or other region shaped to hold a liquid, gel, paste, or solid composition. In some embodiments, a dosage form described elsewhere herein is formulated to be incorporated in a formed object. Incorporation can occur at the point of manufacture of the formed object or prior to final distribution to an end user. In some embodiments, incorporate occurs at the point of use. In some embodiments, the dosage form is provided separately from the formed object and can be incorporated with the formed object at the point of use.

### Methods of Reducing Halitosis in a Companion Animal

The engineered bacteria, formulations thereof, and/or articles containing the engineered bacteria or formulations thereof can be used to reduce halitosis in a companion animal such as a dog or cat. In some embodiments, a liquid formulation can be administered directly to the oral cavity or can be provided to a water source, such as drinking water, where the animal will self-administer the engineered bacteria by drinking the water. In some embodiments, the engineered bacteria can be administered to the animal by giving the animal an object that contains the engineered bacteria. The object can be a toy as described above. When the animal chews on the object, the animal can cause the transfer of the engineered bacteria from the object to the animal's mouth. In some embodiments, the engineered bacteria can be administered to the animal by giving the animal an edible treat containing the engineered bacteria. As the animal chews the treat the engineered bacteria are transferred to the animal's mouth.

After drinking, chewing, ingestion, or other administration of the engineered bacterial cells, the engineered bacterial cells can produce volatile compounds as previously described. When the volatile compounds are produced in the mouth of the animal, they can result in altering the breath odor to that produced by the volatile compounds. The engineered bacteria described herein can produce volatile compounds in the oral cavity for 1-60 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) minutes, 1-12 hours (e.g. 1, 2, 3, 4, 5, 6, 7, 8, 9, 10, 11, 12), or more than 12 hours. The engineered bacteria can reproduce one or more times in the oral cavity. The engineered bacteria described herein can become part of the commensal oral microbiome. In this way the engineered bacteria can allow for longer breath odor control than conventional chews and treats aimed at freshening a dog or cat's breath.

Described in certain aspects herein is a method of improving the breath of a mammal, the method including administering an engineered bacterium or population thereof as described herein, an expression vector as described herein, or both, or a formulation thereof to the mammal.

In certain example embodiments, the method further includes allowing the engineered bacterium or population thereof to produce a volatile compound.

In certain example embodiments, the mammal is a non-human animal. In certain example embodiments, the mammal is canine or a feline.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Bacteria were sampled from the oral cavity of about 20 healthy dogs have been streaked out on conventional laboratory media plates (R2A). The plates are incubated for 4-7 days. Bacterial colonies are then picked, sampled, and DNA isolated for genotyping species by rDNA sequence. Bacteria species from the genus Brevundimonas, Pseudomonas, Xanthomonas, Deinococcus, and Pedobacter were identified. Selected bacteria colonies were tested for natural resistance to standard antibiotics (e.g., terramycin, gentamicin, and tetracycline. Bacteria without natural resistance were then transformed with vectors (pBBR1 and pME6010) containing the odor transgenes. These bacteria are then assayed for stable maintenance of the plasmid (though additional variations may include chromosomal integration of the transgenes). Expression of the transgenes is assayed by real time-PCR (RT-PCR) and presence of the volatile compounds of mass spectrometry, liquid chromatography, and/or gas chromatography. These assays may be performed from bacteria grown in the lab on conventional media or after administration to pets.

Constructs for nptII:pchBA and nptII: BSMT1 were created. Both have been cloned into a pBBR1 and a pME6010 plasmid.

### Example 2—Isolation and Modification of DOG53 Bacteria

About 400 bacteria from the oral cavity of 12 dogs containing desirable traits (fast growing, non-pathogenic, antibiotic susceptibility, amenable to genetic transformation) were isolated. Of the 20 that met the criterial, the most promising bacteria (was termed “DOG53”) was previously uncharacterized from the genera Pseudomonas. Sequencing of at least 2 isolates (“DOG53” and “DOG95”) was completed using an Illumina platform. Sequences from the isolate from DOG53 are provided in SEQ ID NOs: 12 and 14-48 in the Sequence Listing, which is filed herewith and incorporated by reference as if expressed in its entirety herein. Genome assembly of the two isolate strains was performed using Unicycler version: v0.4.8-beta. (about 80% nucleotide sequence identity to the closest known sequenced genome at NCBI). DOG53 was almost identical to another isolate “DOG91”. FIGS. 1-7 demonstrate genomic characterization and comparison of DOG53 and DOG91 isolates. As shown in FIG. 2, the scaffolds were 100 percent identical and are circular as noted by the Unicycler program. They are reverse complements in terms of their sequences with a different break point when making linear. As shown in FIG. 5, Blast 16s rRNA shows greatest similarity along nearly the entire length (99.1%) to Pseudomonas stutzeri. Syntenic dotplots to various strains:
Pseudomonas stutzeri strain RCH2: https://genomevolution.org/r/lcgla
Pseudomonas stutzeri=strain ATCC 17588; LMG 11199: https://genomevolution.org/r/lcglb
Pseudomonas stutzeri strain DSM 4166: https://genomevolution.org/r/lcglc
Pseudomonas stutzeri strain A1501: https://genomevolution.org/r/lcgld
Comparison to RCH2 and DSM4166: https://genomevolution.org/r/lcgm2

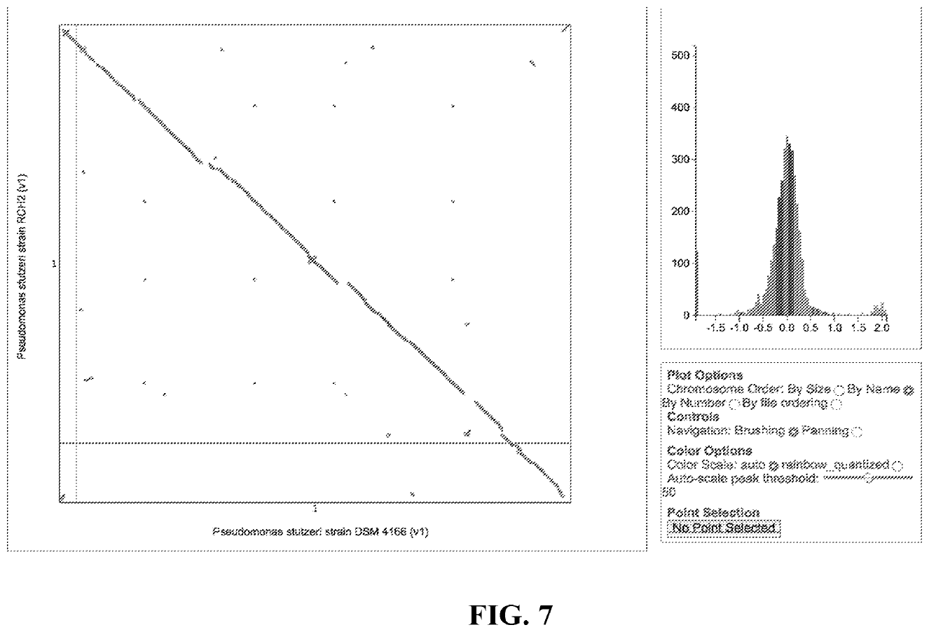

FIG. 6 shows characteristic x-alignments for related bacterial the same genera, but Dog53/Dog91 have DNA sequence and genome structure divergence. Average sequence similarly of syntenic gene pairs between Dog53 is about 80%. This evidences that this is a new species of Pseudomonas. This can be compared to FIG. 7, which shows an example of Example of what two strains from the same species of Pseudomonas looks like.

DOG91 and DOG53 isolated bacteria was transformed with a BSTM1 containing plasmid (see e.g., FIG. 3 and SEQ ID NO: 13) to engineer the bacteria to produce methyl salicylate to yield “DOG91-BSTM1 and “DOG53-BSTM1” engineered bacteria. Presence of plasmid was confirmed by analyzing the engineered strains for BSTM1.

### Example 3—Modified DOG53 Bacteria Produces Mint Volatile Compounds in the Oral Cavity of Dogs

Dog53 containing the BSTM1 plasmid (Dog53-BSTM1) was chosen to be introduced into animals to determine if the engineered bacterium could produce volatile compounds in the oral cavity of animals. Briefly, engineered Dog53-BSTM1 were grown in small batches and reintroduced into the oral cavity of dogs to determine the period these bacteria were present in dog's mouths and if methyl salicylate was produced. This was done in accordance and oversight of the University of Arizona's Institutional Animal Care and Use Committee Program.

Eight dogs were fed about 107 Dog53-BSTM1 bacteria. All subjects had recent vet checkups to ensure that no pre-existing health (oral or systemic) were present. Saliva was collected from the dogs prior to being fed bacteria, immediately after, and then post-feeding at 30 min, 60 min, 90 min, 2 hours, 4 hours, 8 hours, 12 hours, and 24 hours.

The presence and relative quantity of bacteria was determined by culturing on bacteria growth plates, PCR, and qPCR; presence of methyl salicylate by gas chromatography/mass spectrometry using solid-phase microextraction (using pure methyl salicylate as a positive control).

Dog53-BSTM1 bacteria were recovered and detected for up to two hours following feeding, with the relative number of bacteria decreasing over time. Similarly, methyl salicylate was detected as late as two hours after feeding.

### Example 4—Testing of Engineered DOG53 Bacteria

Dog53 bacteria engineered to contain a BSTM1 expression plasmid containing the methyl salicylate production pathway (Dog53-BSTM1 bacteria) were grown in small batches and reintroduced into the oral cavity of dogs to determine the period these bacteria were present in dog's mouths and if methyl salicylate was produced. This was done in accordance with and oversight by the University of Arizona's Institutional Animal Care and Use Committee Program.

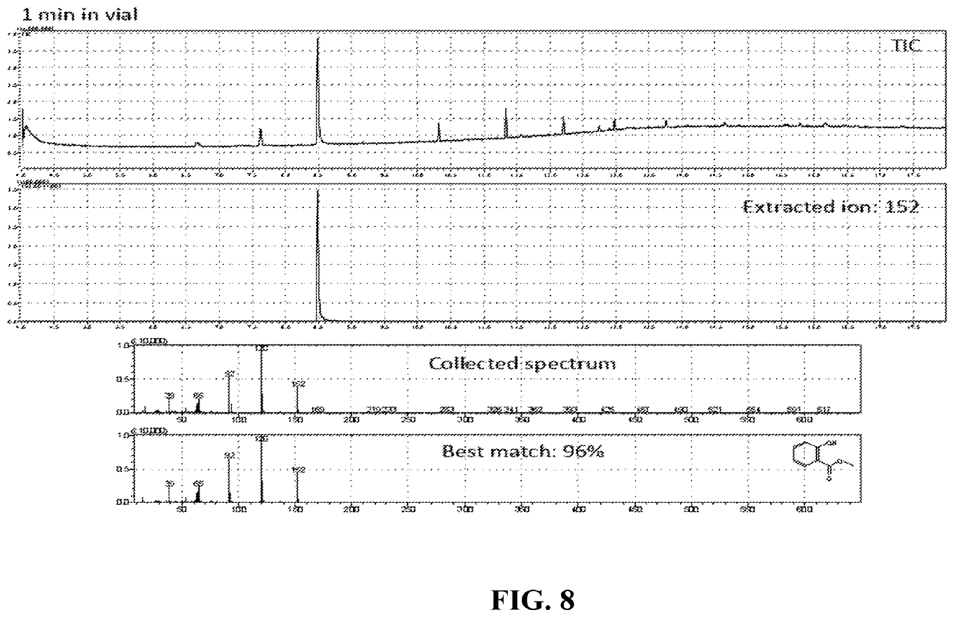

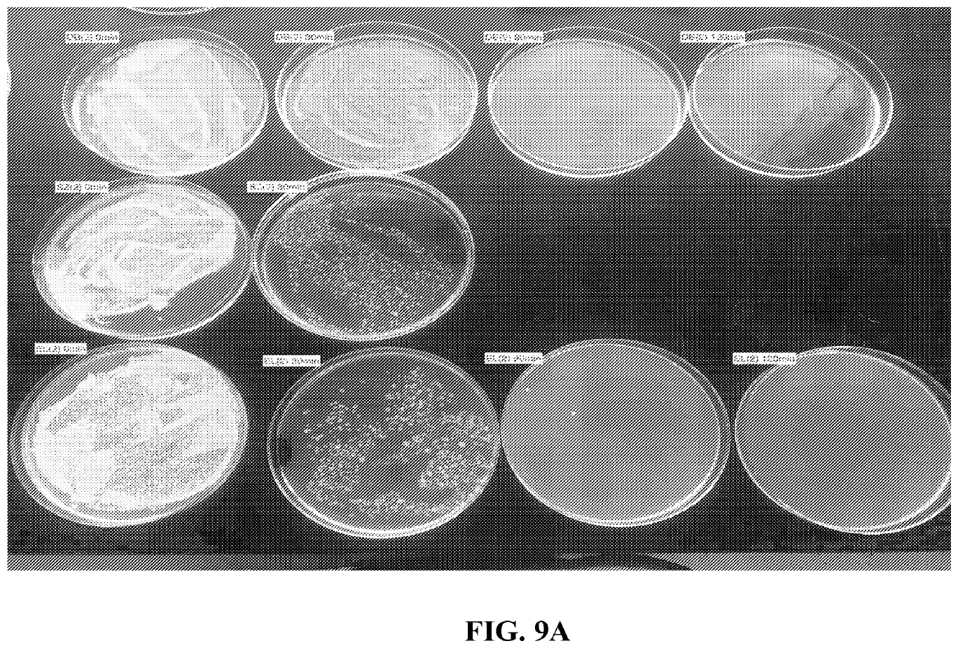

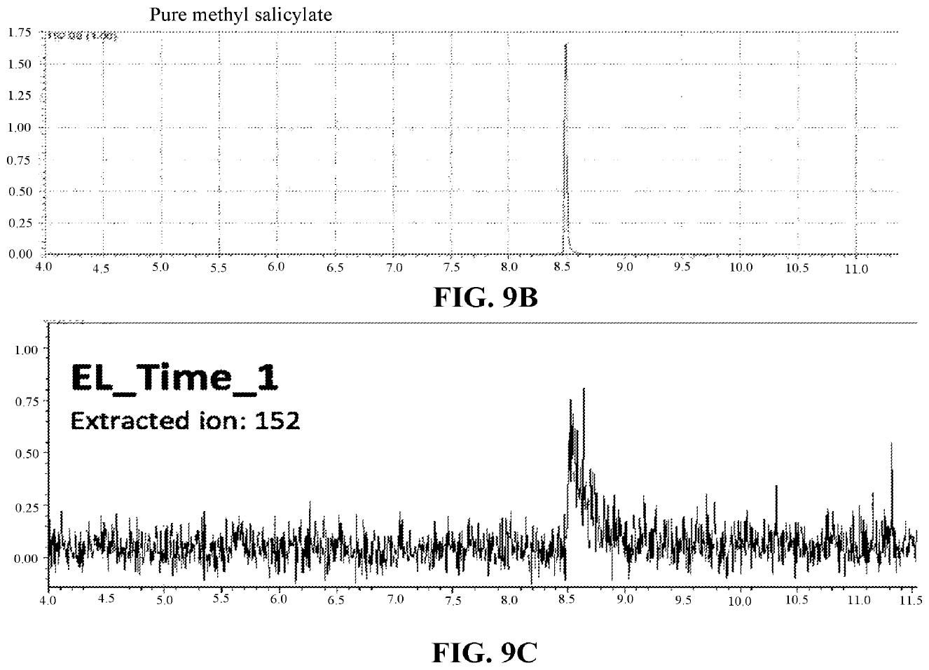

Eight dogs were fed about 107 Dog53-BSTM1 bacteria administered on pet treats (dry biscuits). All subjects had recent vet checkups to ensure that no pre-existing health conditions (oral or systemic) were present. Saliva was collected using sterile cotton swabs from the dogs prior to being fed bacteria, immediately after, and then post-feeding at 30 min, 60 min, 90 min, 2 hours, 4 hours, 8 hours, 12 hours, and 24 hours. Presence and relative quantity of bacteria were determined by culturing on bacteria growth plates, PCR, and qPCR; presence of methyl salicylate was determined by gas chromatography/mass spectrometry using solid-phase microextraction (using pure methyl salicylate as a positive control). Dog53-BSTM1 bacteria were recovered and detected for up to two hours following feeding (FIG. 8), with the relative number of bacteria decreasing over time. Similarly, methyl salicylate was detected as late as 90 min after feeding (FIGS. 9A-9C).

The initial outcome demonstrated in dogs shows the viability of this technology.

### Example 5—Sequences Relevant to the Description and Working Examples

SEQ ID NO: 1-Pseudomonas aeruginosa
pchD, pchC, pchB and pchA genes-
GenBank: X82644.1
(SEQ ID NO: 1)
ATCGGCCTGTGGCGGAGCAATGATGGTGATGGTCATCAGG
TTTTCCTGTAGCCCGGGCGCTGCCGACGCGGCGACGCCTT
CCTGGATACGTGGGGGCAGGGGACTTTTTAGCGGAGATGG
ACAAAGCGCCCTGCACTCCGCCCCTGCAGCGAATGAAAAA
GCCCCGCAATCGAAAGGCGCGGGCTTGCGCGGTCCCTGCC
GCCCGCCAATGATAATAAATCTCATTTCCCAACAATGGCA
ATCGACCGCATCCACGGAGATCGCATGACTTCCTCGCCCG
TCACCCCATCGGCCGTCGACGACGCCCCCGACTGGCCCGC
CGCCTTCGTCCGCCGCTATCTCGACGCCGGCCACTGGCAG
GACCAGAGCTTCGCCGAAGCCCTGGCCACCAGCGCCGCGC
ATCCGCGGCGGATCGCGCTCTGCGACGACGACCAGCGGCT
CAGCTATGCCGACCTGCTCCAGCGCTGCCGCCGCCTGGCA
GCCGGACTGCGCCAGGCCGGCCTCGCACATGGCGACACGG
TAGTGCTGCACCTGCCCAACGGCATCGCCTTCGTCGAGAC
CTGCTTCGCCCTGTTCCAGCTCGGCGTACGCCCGGTGCTC
GCCCTCCCCGCCCATCGCCAGCATGAGATCAGCGGTTTCT
GTCGCTTCGCCGAGGCCAAGGCCTACATCGGCGCCGAACG
GATCGACGGCTTCGACCCACGGCCGATGGCCCGTGAGCTG
CTCGCCAGCGGCGCCTGCCGGATGGCGCTGATCCACGGCG
AAGCCGAGGCGCCGCTGCAAGCGCTGGCACCGCTGTACCA
GGCCGACGCGCTGGAAGACTGCGCCGCCCGCGCCGAGGAT
ATCGCCTGCTTCCAGTTGTCCGGCGGCACCACCGGCACGC
CGAAGCTGATTCCCCGGCGTCATCGCGAGTATCTCTACAA
CGTCCGCGCCAGCGCCGAGGTCTGCGGCTTCGACGAGCAC
ACGGTGTACCTCACCGGCTTGCCGATGGCGCACAACTTCA
CTCTCTGCTGCCCCGGAGTGATCGGCACGCTGCTGGCCAG
CGGCGCGGTGGTGGTCAGCCAGCGCGCCGATCCCGAGCAT
TGCTTCGCCCTGATCGCCCGTGAACGGGTCACCCACACCG
CCCTGGTACCGCCGCTGGCGATGCTCTGGCTGGATGCCCA
GGAAAGCCGCCGCGCCGATCTATCCAGCCTGCGTCTGCTC
CAGGTCGGCGGCTCCAGGCTCGGCAGCAGTGCCGCGCAAC
GCGTCGAACCGGTGCTCGGCTGCCAGTTGCAACAGGTGCT
GGGAATGGCCGAGGGGCTGATCTGCTACACCCGCCTGGAC
GATCCGCCCGAACGCGTGCTGCATACCCAGGGCCGGCCGC
TGTCGCCGGACGACGAAGTGCGGGTGGTCGACGCCGAGGG
CCGCGAGGTCGGCCCCGGCGAGGTGGGCGAACTGACCGTG
CGCGGTCCCTACACCATCCGCGGCTACTACCGCCTGCCTG
AACACAACGCCAAGGCCTTCAGCGCGGACGGCTTCTACCG
CACCGGCGACCGGGTCAGCCGCGACAAGGACGGCTACCTG
GTGGTGGAGGGCCGCGACAAGGACCAGATCAACCGCGGCG
GCGAGAAGATCGCCGCGGAAGAGGTGGAGAACCTGCTGAT
AGCCCATCCGCAGGTGCACGACGCCACCGTCGTGGCGATG
CCCGACAGCCTGCTCGGCGAGCGCACCTGCGCCTTCGTCA
TCCCGCGCCAGCCGGCCGCCTCGGCGCTGAAGCTGAAGCA
ATACCTGCACGCCTGCGGGCTGGCCGCGTTCAAGGTGCCG
GACCGCATCGAGCTGGTCCCGGCCTTCCCCCAAACCGGCA
TCGGCAAGATCAGCAAGAAGGACCTGCGCGAGCGCCTGCG
CCGCGAGCTGGAGGCCCGCGCATGAGCGCCGCCTGGGTCC
GGCCGTTCCGCCTGACGCCGATGCCGCGCCTGCGCCTGGC
CTGCTTCCCCCATGCAGGCGGCAGCGCCAGCTTCTTCCGT
AGCTGGAGCGAACGCCTGCCGCCAGACATCGACCTGCTTG
CCCTGCAGTACCCGGGTCGCGAGGACCGCTTCAACGAGGC
GCCGGCCACCCGCCTGGAGGACCTCGCCGACGGCGCCGCC
CTCGCCCTGCGCGATTTCGCCGACGCGCCCCTGGCGCTGT
TCGGCCACAGTCTCGGCGCGGCGCTGGCCTACGAAACCGC
CCTGCGCCTGGAAACGCCGGCCGCCCTGCGCCACCTGTTC
GTCTCCGCCCATCCGGCACCGCACCGGCAACGCGGCGGCG
CGTTGCACCGCGGCGACGAGGCGGCGCTGCTGGAGGACGT
CCGCCGCCAGGGTGGCGCCAGCGAGCTACTCGAGGACGCC
GACCTGCGCGCGCTGTTCCTGCCGATCCTGCGCGCCGACT
ACCAGGCGATCGAGACCTACCGACGGGCGCAGCCCATCGC
CCTGGCCTGCGCCCTCGACGTCCTCCTCGGCGAGCACGAC
GAGGAAGTCAGCGCCGCCGAGGCGCAGGCCTGGAGCGACG
CCAGCCGGACTCCCGCCAGGCTGCGGCGCTTTCCTGGCGG
CCACTTCTACCTGAGCGAGGGGCGCGACGCGGTGATCGAG
CACCTGCTGCGCCGCCTCGCACATCCCGACGCCCTTTCCC
GAGAGGTTGCATGATGAAAACTCCCGAAGACTGCACCGGC
CTGGCGGACATCCGCGAGGCCATCGACCGGATCGACCTGG
ATATCGTCCAGGCCCTCGGCCGCCGCATGGACTACGTCAA
GGCGGCGTCGCGCTTCAAGGCCAGCGAGGCGGCGATTCCG
GCGCCCGAGCGGGTCGCCGCGATGCTCCCCGAGCGCGCCC
GCTGGGCCGAGGAAAACGGACTCGACGCGCCCTTCGTCGA
GGGACTGTTCGCGCAGATCATCCACTGGTACATCGCCGAG
CAGATCAAGTACTGGCGCCAGACACGGGGTGCCGCATGAG
CCGGCTGGCGCCCCTGAGCCAGTGCCTGCACGCCTTGCGC
GGCACCTTCGAGCGCGCCATCGGCCAGGCGCAGGCGCTCG
ATCGTCCGGTGCTGGTGGCGGCATCGTTCGAGATCGACCC
ATTGGACCCGCTGCAGGTATTCGGTGCCTGGGACGACCGG
CAAACGCCCTGCCTGTACTGGGAACAGCCCGAGCTGGCGT
TCTTCGCCTGGGGCTGCGCCCTGGAGCTGCAAGGCCACGG
CGAACAGCGCTTCGCCCGGATCGAGGAAAACTGGCAATTG
CTCTGCGCCGACGCCGTGGTCGAGGGCCCGCTGGCGCCGC
GCCTGTGCGGCGGATTCCGCTTCGATCCGCGCGGCCCGCG
CGAGGAACACTGGCAAGCCTTCGCCGATGCCAGCCTGATG
CTCGCCGGCATCACCGTGCTGCGCGAGGGCGAACGCTACC
GGGTACTCTGCCAACACCTGGCCAAGCCCGGCGAAGATGC
CCTGGCCCTGGCCGCCTACCACTGCTCGGCGCTACTGCGC
CTGAGGCAGCCGGCCAGACGCCGGCCCTCGGGGCCGACCG
CTGGCGCGCAGGGCGACGCTTCGGCGCAGGAGCGCAGGCA
ATGGGAAGCCAAGGTGAGCGACGCGGTAAGCAGTGTCCGC
CAGGGACGCTTCGGCAAGGTCGTGCTGGCCCGCACCCAGG
CCCGGCCTCTCGGCGACATCGAGCCGTGGCAGGTCATCGA
ACACCTGCGTCTGCAACATGCCGACGCCCAGCTGTTCGCC
TGTCGCCGCGGCAACGCCTGCTTCCTCGGCGCCTCCCCGG
AACGCCTGGTCCGCATTCGCGCCGGCGAGGCACTCACCCA
TGCCCTGGCCGGGACCATCGCCCGCGGCGGCGATGCCCAG
GAAGATGCGCGGCTCGGACAGGCCCTGCTGGACAGCGCCA
AGGACAGGCACGAACACCAGTTGGTGGTGGAGGCGATCCG
TACGGCCCTGGAACCCTTCAGCGAGGTGCTGGAAATCCCC
GATGCGCCCGGCCTGAAACGACTGGCGCGAGTCCAGCACC
TGAACACGCCGATCCGCGCCCGCCTCGCTGACGCAGGCGG
CATCCTGCGGCTGCTACAAGCGCTGCATCCGACCCCCGCG
GTGGGCGGCTACCCACGCAGCGCGGCGCTGGACTACATCC
GCCAGCACGAAGGGATGGACCGCGGCTGGTACGCCGCGCC
GCTGGGCTGGCTCGACGGCGAAGGCAACGGCGATTTCCTG
GTGGCGCTGCGCTCGGCCCTGCTCACGCCGGGCCGGGGCT
ACCTGTTCGCCGGCTGCGGTCTGGTAGGCGATTCGGAACC
GGCCCACGAGTATCGCGAAACCTGCCTTAAGCTCAGTGCC
ATGCGGGAAGCTCTATCCGCCATAGGCGGCCTGGACGAAG
TGCCCTTGCAGCGCGGCGTCGCCTGAAAACGAAGACCCCC
TGCGGCCCGAGGGCGGCAGGGGGTCTTCGAAGGTCTGCCC
GTGGCGCGGACGGGCCGCCGCAAGGGCGTCGCTTAGAACG
GAATGTCGTCGTCGAAGCTGTCGTAGTCCTGGGCCGGTTG
CGGCGCCGGTTGCTGCTGCGGGGCCGGACGCGACTGCTGC
TGCGGGGCCTGCTGCGGGCGCTGCATGGGCTCGCGGGGCG
CGCTGCGAATCGTCGCCGGAGGGGCGGCCGCCGAGCAACT
GCATGTTGCCGTTGATGTCGAC
SEQ ID NO: 2-AY233465.1 Petunia x hybrida
S-adenosyl-L-methionine: benzoic acid/
salicylic acid carboxyl methyltransferase
(BSMT1) mRNA, complete cds
(SEQ ID NO: 2)
AAAGATCAAGAAAGAGATACTCATAGCAAGAAGAAATGGA
AGTTGTTGAAGTTCTTCACATGAATGGAGGAAATGGAGAC
AGTAGCTATGCAAACAATTCTTTGGTTCAGCAAAAGGTGA
TTCTCATGACAAAGCCAATAACTGAGCAAGCCATGATTGA
TCTCTACAGCAGCCTCTTTCCAGAAACCTTATGCATTGCA
GATTTGGGTTGTTCTTTGGGAGCTAACACTTTCTTGGTGG
TCTCACAGCTTGTTAAAATAGTAGAAAAAGAACGAAAAAA
GCATGGTTTTAAGTCTCCAGAGTTTTATTTTCACTTCAAT
GATCTTCCTGGCAATGATTTTAATACACTTTTTCAGTCAC
TGGGGGCATTTCAAGAAGATTTGAGAAAGCATATAGGGGA
AAGCTTTGGTCCATGTTTTTTCAGTGGAGTGCCTGGTTCA
TTTTATACTAGACTTTTCCCTTCCAAAAGTTTACATTTTG
TTTACTCCTCCTACAGTCTCATGTGGCTATCTCAGGTGCC
TAATGGGATTGAAAATAACAAGGGAAACATTTACATGGCA
AGAACAAGCCCTCTAAGTGTTATTAAAGCATACTACAAGC
AATATGAAATAGATTTTTCAAATTTTCTCAAGTACCGTTC
AGAGGAATTGATGAAAGGTGGAAAGATGGTGTTAACACTC
CTAGGTAGAGAAAGTGAGGATCCTACTAGCAAAGAATGCT
GTTACATTTGGGAGCTTCTAGCCATGGCCCTCAATAAGTT
GGTTGAAGAGGGATTGATAAAAGAAGAGAAAGTAGATGCA
TTCAATATTCCTCAATACACACCATCACCAGCAGAAGTAA
AGTACATAGTTGAGAAGGAAGGATCATTCACCATTAATCG
CTTGGAAACATCAAGAGTTCATTGGAATGCTTCTAATAAT
GAGAAGAATGGTGGTTACAATGTGTCAAGGTGCATGAGAG
CTGTGGCTGAGCCTTTGCTTGTCAGCCACTTTGACAAGGA
ATTGATGGATTTAGTGTTCCACAAGTACGAAGAGATTGTT
TCTGATTGCATGTCCAAAGAGAATACTGAGTTTATAAATG
TCATCATCTCCTTGACCAAAATAAATTAAGGCACTCAATG
TCTATTTTCGGTCGAAATCCGGTGGTCGAAAAACCCGAAT
ATTTTAGTAGTGATCCGATATAAACTACATATAAATATTA
CAATAGATTAAAATACATATATGTTTAATAAGTTCACGCA
CATATTGAATTATCTTATTCAAATGTTTAATACGACTATG
CTGAATGTACCAATTCTCTATTGTTGTTTTGCACTTCATA
GGTGGTCGTGGTCGAGGTGCTAATTATTTATCTAGTCATT
GTCTTGTACACTAAAGGTTCTGTACCACAGGGAATTATCT
CATGCATGTCTTTATTATATATTTAAATGGATAAGTTACT
GCCTTGATTTTAAAAAAAAAAAAAAAAAAAAAA
SEQ ID NO: 3-Pseudomonas aeruginosa pchA
(GenBank Accession No.: X82644.1,
UniProtKB ID: Q51508)
(SEQ ID NO: 3)
MSRLAPLSQCLHALRGTFERAIGQAQALDRPVLVAASFEI
DPLDPLQVFGAWDDRQTPCLYWEQPELAFFAWGCALELQG
HGEQRFARIEENWQLLCADAVVEGPLAPRLCGGFRFDPRG
PREEHWQAFADASLMLAGITVLREGERYRVLCQHLAKPGE
DALALAAYHCSALLRLRQPARRRPSGPTAGAQGDASAQER
RQWEAKVSDAVSSVRQGRFGKVVLARTQARPLGDIEPWQV
IEHLRLQHADAQLFACRRGNACFLGASPERLVRIRAGEAL
THALAGTIARGGDAQEDARLGQALLDSAKDRHEHQLVVEA
IRTALEPFSEVLEIPDAPGLKRLARVQHLNTPIRARLADA
GGILRLLQALHPTPAVGGYPRSAALDYIRQHEGMDRGWYA
APLGWLDGEGNGDFLVALRSALLTPGRGYLFAGCGLVGDS
EPAHEYRETCLKLSAMREALSAIGGLDEVPLQRGVA
SEQ ID NO: 4-Pseudomonas aeruginosa
pchB-UniProtKB ID: Q51507
(SEQ ID NO: 4)
MMKTPEDCTGLADIREAIDRIDLDIVQALGRRMDYVKAAS
RFKASEAAIPAPERVAAMLPERARWAEENGLDAPFVEGLF
AQIIHWYIAEQIKYWRQTRGAA
SEQ ID NO: 5-NCBI Reference Sequence:
WP_003114686.1 (ACCESSION WP_003114686)
WP_003114686.1 MULTISPECIES: salicylate
biosynthesis isochorismate
synthase [Pseudomonas]
(PchA)
(SEQ ID NO: 5)
MSRLAPLSQCLHALRGTFERAIGQAQALDRPVLVAASFEI
DPLDPLQVFGAWDDRQTPCLYWEQPELAFFAWGCALELQG
HGEQRFARIEENWQLLCADAVVEGPLAPRLCGGFRFDPRG
PREEHWQAFADASLMLAGITVLREGERYRVLCQHLAKPGE
DALALAAYHCSALLRLRQPARRRPSGPTAGAQGDASAQER
RQWEAKVSDAVSSVRQGRFGKVVLARTQARPLGDIEPWQV
IEHLRLQHADAQLFACRRGNACFLGASPERLVRIRAGEAL
THALAGTIARGGDAQEDARLGQALLDSAKDRHEHQLVVEA
IRTALEPFSEVLEIPDAPGLKRLARVQHLNTPIRARLADA
GGILRLLQALHPTPAVGGYPRSAALDYIRQHEGMDRGWYA
APLGWLDGEGNGDFLVALRSALLTPGRGYLFAGCGLVGDS
EPAHEYRETCLKLSAMREALSAIGGLDEVPLQRGVA
SEQ ID NO: 6-pchB [Pseudomonas aeruginosa] PAO1
GenBank: CAA57968.1
(SEQ ID NO: 6)
MMKTPEDCTGLADIREAIDRIDLDIVQALGRRMDYVKAAS
RFKASEAAIPAPERVAAMLPERARWAEENGLDAPFVEGLF
AQIIHWYIAEQIKYWRQTRGAA
SEQ ID NO: 7-S-adenosyl-L-methionine:
benzoic acid/salicylic acid carboxyl
methyltransferase [Petunia x hybrida]
GenBank: AAO45012.1
(BSMT1)
(SEQ ID NO: 7)
MEVVEVLHMNGGNGDSSYANNSLVQQKVILMTKPITEQAM
IDLYSSLFPETLCIADLGCSLGANTFLVVSQLVKIVEKER
KKHGFKSPEFYFHFNDLPGNDFNTLFQSLGAFQEDLRKHI
GESFGPCFFSGVPGSFYTRLFPSKSLHFVYSSYSLMWLSQ
VPNGIENNKGNIYMARTSPLSVIKAYYKQYEIDFSNFLKY
RSEELMKGGKMVLTLLGRESEDPTSKECCYIWELLAMALN
KLVEEGLIKEEKVDAFNIPQYTPSPAEVKYIVEKEGSFTI
NRLETSRVHWNASNNEKNGGYNVSRCMRAVAEPLLVSHFD
KELMDLVFHKYEEIVSDCMSKENTEFINVIISLTKIN
SEQ ID NO: 8: Arabidopsis thaliana
S-adenosyl-L-methionine-dependent
methyltransferases superfamily protein
(BSMT1), mRNA, NCBI Reference Sequence:
NM_111981.5
(SEQ ID NO: 8)
CTTGATGAAGTTCCCTCTCTCTATAAATTGAAGTGTGTGA
GATGCATCATCCATACACAACATCTTCCATCTTCCGATAA
TTCTCCTTTAGTCTTATAATCACTAATAAGTACGATAATT
GAAATGGATCCAAGATTCATCAACACCATTCCTTCCTTGA
GGTATGATGATGATAAGTGTGATGATGAATATGCGTTTGT
GAAAGCTCTATGTATGAGTGGTGGAGATGGTGCCAACAGT
TACTCCGCCAATTCTCGCCTTCAGAAAAAAGTTTTATCAA
TGGCCAAACCAGTCTTGGTAAGAAACACAGAAGAAATGAT
GATGAACTTAGACTTTCCTACGTACATCAAAGTTGCTGAA
TTGGGTTGTTCTTCGGGACAAAACTCTTTTTTGGCTATCT
TTGAGATCATCAACACCATTAATGTGTTGTGCCAACATGT
GAACAAAAACTCACCAGAGATCGATTGTTGTCTAAACGAT
CTCCCGGAAAATGATTTCAACACGACCTTTAAGTTCGTAC
CTTTCTTCAACAAGGAGCTCATGATCACAAACAAATCATC
ATGTTTCGTCTATGGAGCTCCAGGTTCCTTCTATTCCAGG
CTCTTCTCTCGCAATAGCCTCCATTTAATACATTCCTCTT
ATGCACTCCATTGGCTCTCTAAGGTTCCCGAGAAACTTGA
GAATAATAAGGGGAATCTGTACATAACAAGTTCAAGTCCT
CAAAGTGCATACAAGGCCTACTTGAATCAATTCCAAAAAG
ACTTCACCATGTTTCTAAGGTTACGTTCTGAAGAAATTGT
CTCTAATGGACGTATGGTTCTCACCTTCATCGGAAGAAAC
ACTCTTAACGATCCATTGTATAGAGATTGTTGTCACTTTT
GGACATTGCTATCAAACTCTCTCCGTGACCTAGTCTTTGA
GGGTCTTGTGAGTGAGTCAAAGCTGGACGCATTCAACATG
CCGTTTTATGATCCGAACGTACAAGAACTCAAAGAAGTGA
TACAAAAAGAGGGCTCTTTTGAAATCAATGAATTGGAGTC
ACATGGATTTGATCTTGGTCACTACTACGAAGAAGATGAC
TTTGAAGCAGGACGCAATGAAGCTAATGGCATAAGAGCTG
TTAGTGAACCAATGCTCATTGCTCATTTTGGAGAAGAAAT
TATCGATACCTTGTTCGATAAGTATGCATACCATGTGACT
CAACATGCCAACTGCAGGAACAAAACGACTGTCAGTCTTG
TCGTTTCCTTGACTAAGAAGTAAGAAGTAATCAACTTCTG
TCATGTTGCTCTATTTGTATTTATTTACTACTGTTATTTT
GTTTCCTTGAATAAATTTCAACACCTGCCATTCAATGTGA
TGATGTGGAGCTAGTATTGGAAAAAAGTGTGACTGTAATA
TATTATGGTAAATTTGGTAAATGATTTATTTCCTGCAAAA
GTTGCAGATACTGCTTTCAAGTATGAAG
SEQ ID NO: 9: Arabidopsis thaliana
S-adenosyl-L-methionine-dependent
methyltransferases superfamily protein
(BSMT1), polypeptide translated
from SEQ ID NO: 8
(SEQ ID NO: 9)
MDPRFINTIPSLRYDDDKCDDEYAFVKALCMSGGDGANSY
SANSRLQKKVLSMAKPVLVRNTEEMMMNLDFPTYIKVAEL
GCSSGQNSFLAIFEIINTINVLCQHVNKNSPEIDCCLNDL
PENDFNTTFKFVPFFNKELMITNKSSCFVYGAPGSFYSRL
FSRNSLHLIHSSYALHWLSKVPEKLENNKGNLYITSSSPQ
SAYKAYLNQFQKDFTMFLRLRSEEIVSNGRMVLTFIGRNT
LNDPLYRDCCHFWTLLSNSLRDLVFEGLVSESKLDAFNMP
FYDPNVQELKEVIQKEGSFEINELESHGFDLGHYYEEDDF
EAGRNEANGIRAVSEPMLIAHFGEEIIDTLFDKYAYHVTQ
HANCRNKTTVSLVVSLTKK
SEQ ID NO: 10-phcBA polynucleotide
sequence with Gateway cloning vector
recombinase sites on the 5′ and 3′
ends (about 30 bp each)
(SEQ ID NO: 10)
GGGGACAAGTTTGTACAAAAAAGCAGGCTCCCCGGAATTG
CCAGCTGGGGCGCCCTCTGGTAAGGTTGGGAAGCCCTGCA
AATGAAAACTCCTGAAGATTGCACCGGACTGGCAGATATC
CGTGAGGCAATCGATCGGATCGATCTGGATATCGTACAGG
CACTCGGACGTCGTATGGATTACGTAAAGGCAGCATCGCG
TTTCAAGGCAAGCGAGGCAGCAATACCAGCACCTGAGCGG
GTAGCAGCAATGCTCCCTGAGCGTGCACGTTGGGCAGAGG
AAAACGGACTCGATGCACCTTTCGTAGAGGGACTGTTCGC
ACAGATCATCCACTGGTACATCGCAGAGCAGATCAAGTAC
TGGCGTCAGACACGGGGTGCAGCATGAGCCGGCTGGCACC
TCTGAGCCAGTGCCTGCACGCATTGCGTGGAACCTTCGAA
CGTGCAATCGGACAGGCACAGGCACTAGATCGTCCGGTGC
TGGTGGCAGCATCGTTCGAAATCGATCCATTGGATCCGCT
ACAGGTATTCGGTGCATGGGATGATCGGCAAACGCCTTGC
CTGTACTGGGAACAGCCTGAACTGGCATTCTTCGCATGGG
GATGCGCACTGGAACTGCAAGGACACGGAGAACAGCGTTT
CGCACGGATCGAAGAAAACTGGCAATTGCTATGCGCAGAT
GCAGTGGTAGAAGGACCGCTGGCACCGCGTCTGTGCGGAG
GATTCCGTTTCGATCCGCGTGGACCGCGTGAAGAACACTG
GCAAGCATTCGCAGATGCAAGCCTGATGCTAGCAGGAATC
ACCGTGCTGCGTGAAGGAGAACGTTACCGGGTACTATGCC
AACACCTGGCAAAGCCTGGAGAAGATGCACTGGCACTGGC
AGCATACCATTGCTCGGCACTACTGCGTCTGAGGCAGCCG
GCAAGACGTCGGCCATCGGGTCCGACCGCTGGAGCACAGG
GAGATGCTTCGGCACAGGAACGTAGGCAATGGGAAGCAAA
GGTGAGCGATGCAGTAAGCAGTGTACGTCAGGGACGTTTC
GGAAAGGTAGTACTGGCACGAACCCAGGCACGGCCTCTAG
GAGATATCGAACCGTGGCAGGTAATCGAACACCTGCGTCT
GCAACATGCAGATGCACAGCTGTTCGCATGTCGTCGTGGA
AACGCATGCTTCCTAGGAGCCTCCCCGGAACGTCTGGTAC
GTATTCGTGCAGGAGAAGCACTAACCCATGCACTGGCAGG
TACCATCGCACGTGGAGGAGATGCACAGGAAGATGCACGG
CTAGGACAGGCACTGCTGGATAGCGCAAAGGATAGGCACG
AACACCAGTTGGTGGTGGAAGCAATCCGTACGGCACTGGA
ACCTTTCAGCGAAGTGCTGGAAATCCCTGATGCACCTGGA
CTGAAACGACTGGCACGAGTACAGCACCTGAACACGCCGA
TCCGTGCACGTCTAGCTGATGCAGGAGGCATCCTGCGGCT
GCTACAAGCACTGCATCCGACCCCTGCAGTGGGAGGCTAC
CCACGTAGCGCAGCACTGGATTACATCCGTCAGCACGAAG
GTATGGATCGTGGATGGTACGCAGCACCGCTGGGATGGCT
AGATGGAGAAGGAAACGGAGATTTCCTGGTGGCACTGCGT
TCGGCACTGCTAACGCCGGGACGGGGATACCTGTTCGCAG
GCTGCGGTCTGGTAGGAGATTCGGAACCGGCACACGAATA
TCGTGAAACCTGCCTTAAGCTAAGTGCAATGCGGGAAGCT
CTATCCGCAATAGGAGGCCTGGATGAAGTGCCTTTGCAGC
GTGGAGTAGCATAATAACACCCAGCTTTCTTGTACAAAGT
GGTCCCC
SEQ ID NO: 11-BSMT1 polynucleotide sequence
with Gateway cloning vector recombinase sites
on the 5′ and 3′ ends about 30 bp each)
(SEQ ID NO: 11)
GGGGACAAGTTTGTACAAAAAAGCAGGCTCCCCGGAATTG
CCAGCTGGGGCGCCCTCTGGTAAGGTTGGGAAGCCCTGCA
AATGGAAGTTGTTGAAGTTCTTCACATGAATGGAGGAAAT
GGAGACAGTAGCTATGCAAACAATTCTTTGGTTCAGCAAA
AGGTGATTCTCATGACAAAGCCAATAACTGAGCAAGCCAT
GATTGATCTCTACAGCAGCCTCTTTCCAGAAACCTTATGC
ATTGCAGATTTGGGTTGTTCTTTGGGAGCTAACACTTTCT
TGGTGGTCTCACAGCTTGTTAAAATAGTAGAAAAAGAACG
AAAAAAGCATGGTTTTAAGTCTCCAGAGTTTTATTTTCAC
TTCAATGATCTTCCTGGCAATGATTTTAATACACTTTTTC
AGTCACTGGGGGCATTTCAAGAAGATTTGAGAAAGCATAT
AGGGGAAAGCTTTGGTCCATGTTTTTTCAGTGGAGTGCCT
GGTTCATTTTATACTAGACTTTTCCCTTCCAAAAGTTTAC
ATTTTGTTTACTCCTCCTACAGTCTCATGTGGCTATCTCA
GGTGCCTAATGGGATTGAAAATAACAAGGGAAACATTTAC
ATGGCAAGAACAAGCCCTCTAAGTGTTATTAAAGCATACT
ACAAGCAATATGAAATAGATTTTTCAAATTTTCTCAAGTA
CCGTTCAGAGGAATTGATGAAAGGTGGAAAGATGGTGTTA
ACACTCCTAGGTAGAGAAAGTGAGGATCCTACTAGCAAAG
AATGCTGTTACATTTGGGAGCTTCTAGCCATGGCCCTCAA
TAAGTTGGTTGAAGAGGGATTGATAAAAGAAGAGAAAGTA
GATGCATTCAATATTCCTCAATACACACCATCACCAGCAG
AAGTAAAGTACATAGTTGAGAAGGAAGGATCATTCACCAT
TAATCGCTTGGAAACATCAAGAGTTCATTGGAATGCTTCT
AATAATGAGAAGAATGGTGGTTACAATGTGTCAAGGTGCA
TGAGAGCTGTGGCTGAGCCTTTGCTTGTCAGCCACTTTGA
CAAGGAATTGATGGATTTAGTGTTCCACAAGTACGAAGAG
ATTGTTTCTGATTGCATGTCCAAAGAGAATACTGAGTTTA
TAAATGTCATCATCTCCTTGACCAAAATAAATTAACACCC
AGCTTTCTTGTACAAAGTGGTCCCC
SEQ ID NO: 12-16s rRNA sequence from
Dog53: >Pseudomonas sp.
(Dog53-PROKKA_03272020.gff, Annotated
by prokka 1.12 available through CyVerse
Discovery Environment, v1),
Location: 216-1634 (length: 1419),
Chromosome: 18, Strand: 1
(SEQ ID NO: 12)
ATGGCTCAGATTGA
ACGCTGGCGGCAGGCCTAACACATGCAAGTCGAGCGGATG
AGTGGAGCTTGCTCCATGATTCAGCGGCGGACGGGTGAGT
AATGCCTAGGAATCTGCCTGGTAGTGGGGGACAACGTTTC
GAAAGGAACGCTAATACCGCATACGTCCTACGGGAGAAAG
CAGGGGACCTTCGGGCCTTGCGCTATCAGATGAGCCTAGG
TCGGATTAGCTAGTTGGTGAGGTAATGGCTCACCAAGGCG
ACGATCCGTAACTGGTCTGAGAGGATGATCAGTCACACTG
GAACTGAGACACGGTCCAGACTCCTACGGGAGGCAGCAGT
GGGGAATATTGGACAATGGGCGAAAGCCTGATCCAGCCAT
GCCGCGTGTGTGAAGAAGGTCTTCGGATTGTAAAGCACTT
TAAGTTGGGAGGAAGGGCAGTAAGTTAATACCTTGCTGTT
TTGACGTTACCGACAGAATAAGCACCGGCTAACTTCGTGC
CAGCAGCCGCGGTAATACGAAGGGTGCAAGCGTTAATCGG
AATTACTGGGCGTAAAGCGCGCGTAGGTGGTTTGTTAAGT
TGGATGTGAAAGCCCCGGGCTCAACCTGGGAACTGCATCC
AAAACTGGCAAGCTAGAGTATGGCAGAGGGTGGTGGAATT
TCCTGTGTAGCGGTGAAATGCGTAGATATAGGAAGGAACA
CCAGTGGCGAAGGCGACCACCTGGGCTAATACTGACACTG
AGGTGCGAAAGCGTGGGGAGCAAACAGGATTAGATACCCT
GGTAGTCCACGCCGTAAACGATGTCGACTAGCCGTTGGGA
TCCTTGAGATCTTAGTGGCGCAGCTAACGCATTAAGTCGA
CCGCCTGGGGAGTACGGCCGCAAGGTTAAAACTCAAATGA
ATTGACGGGGGCCCGCACAAGCGGTGGAGCATGTGGTTTA
ATTCGAAGCAACGCGAAGAACCTTACCAGGCCTTGACATG
CAGAGAACTTTCCAGAGATGGATTGGTGCCTTCGGGAACT
CTGACACAGGTGCTGCATGGCTGTCGTCAGCTCGTGTCGT
GAGATGTTGGGTTAAGTCCCGTAACGAGCGCAACCCTTGT
CCTTAGTTACCAGCACGTGATGGTGGGCACTCTAAGGAGA
CTGCCGGTGACAAACCGGAGGAAGGTGGGGATGACGTCAA
GTCATCATGGCCCTTACGGCCTGGGCTACACACGTGCTAC
AATGGTCGGTACAAAGGGTTGCCAAGCCGCGAGGTGGAGC
TAATCCCATAAAACCGATCGTAGTCCGGATCGCAGTCTGC
AACTCGACTGCGTGAAGTCGGAATCGCTAGTAATCGTGAA
TCAGAATGTCACGGTGAATACGTTCCCGGGCCTTGTACAC
ACCGCCCGTCACACCATGGGAGTGGGTTGCTCCAGAAGTA
GCTAG
SEQ ID NO: 13-Mint Plasmid
(SEQ ID NO: 13)
GGGCAACGTGGCGGCCAGTCTCAAGCACGCCTACCGCGAG
CGCGAGACGCCCAACGCTGACGCCAGCAGGACGCCAGAGA
ACGAGCACTGGGCGGCCAGCAGCACCGATGAAGCGATGGG
CCGACTGCGCGAGTTGCTGCCAGAGAAGCGGCGCAAGGAC
GCTGTGTTGGCGGTCGAGTACGTCATGACGGCCAGCCCGG
AATGGTGGAAGTCGGCCAGCCAAGAACAGCAGGCGGCGTT
CTTCGAGAAGGCGCACAAGTGGCTGGCGGACAAGTACGGG
GCGGATCGCATCGTGACGGCCAGCATCCACCGTGACGAAA
CCAGCCCGCACATGACCGCGTTCGTGGTGCCGCTGACGCA
GGACGGCAGGCTGTCGGCCAAGGAGTTCATCGGCAACAAA
GCGCAGATGACCCGCGACCAGACCACGTTTGCGGCCGCTG
TGGCCGATCTAGGGCTGCAACGGGGCATCGAGGGCAGCAA
GGCACGTCACACGCGCATTCAGGCGTTCTACGAGGCCCTG
GAGCGGCCACCAGTGGGCCACGTCACCATCAGCCCGCAAG
CGGTCGAGCCACGCGCCTATGCACCGCAGGGATTGGCCGA
AAAGCTGGGAATCTCAAAGCGCGTTGAGACGCCGGAAGCC
GTGGCCGACCGGCTGACAAAAGCGGTTCGGCAGGGGTATG
AGCCTGCCCTACAGGCCGCCGCAGGAGCGCGTGAGATGCG
CAAGAAGGCCGATCAAGCCCAAGAGACGGCCCGAGACCTT
CGGGAGCGCCTGAAGCCCGTTCTGGACGCCCTGGGGCCGT
TGAATCGGGATATGCAGGCCAAGGCCGCCGCGATCATCAA
GGCCGTGGGCGAAAAGCTGCTGACGGAACAGCGGGAAGTC
CAGCGCCAGAAACAGGCCCAGCGCCAGCAGGAACGCGGGC
GCGCACATTTCCCCGAAAAGTGCCACCTGGGATGAATGTC
AGCTACTGGGCTATCTGGACAAGGGAAAACGCAAGCGCAA
AGAGAAAGCAGGTAGCTTGCAGTGGGCTTACATGGCGATA
GCTAGACTGGGCGGTTTTATGGACAGCAAGCGAACCGGAA
TTGCCAGCTGGGGCGCCCTCTGGTAAGGTTGGGAAGCCCT
GCAAAGTAAACTGGATGGCTTTCTTGCCGCCAAGGATCTG
ATGGCGCAGGGGATCAAGATCTGATCAAGAGACAGGATGA
GGATCGTTTCGCATGATTGAACAAGATGGATTGCACGCAG
GTTCTCCGGCCGCTTGGGTGGAGAGGCTATTCGGCTATGA
CTGGGCACAACAGACAATCGGCTGCTCTGATGCCGCCGTG
TTCCGGCTGTCAGCGCAGGGGCGCCCGGTTCTTTTTGTCA
AGACCGACCTGTCCGGTGCCCTGAATGAACTGCAGGACGA
GGCAGCGCGGCTATCGTGGCTGGCCACGACGGGCGTTCCT
TGCGCAGCTGTGCTCGACGTTGTCACTGAAGCGGGAAGGG
ACTGGCTGCTATTGGGCGAAGTGCCGGGGCAGGATCTCCT
GTCATCTCACCTTGCTCCTGCCGAGAAAGTATCCATCATG
GCTGATGCAATGCGGCGGCTGCATACGCTTGATCCGGCTA
CCTGCCCATTCGACCACCAAGCGAAACATCGCATCGAGCG
AGCACGTACTCGGATGGAAGCCGGTCTTGTCGATCAGGAT
GATCTGGACGAAGAGCATCAGGGGCTCGCGCCAGCCGAAC
TGTTCGCCAGGCTCAAGGCGCGCATGCCCGACGGCGAGGA
TCTCGTCGTGACCCATGGCGATGCCTGCTTGCCGAATATC
ATGGTGGAAAATGGCCGCTTTTCTGGATTCATCGACTGTG
GCCGGCTGGGTGTGGCGGACCGCTATCAGGACATAGCGTT
GGCTACCCGTGATATTGCTGAAGAGCTTGGCGGCGAATGG
GCTGACCGCTTCCTCGTGCTTTACGGTATCGCCGCTCCCG
ATTCGCAGCGCATCGCCTTCTATCGCCTTCTTGACGAGTT
CTTCTGAGCGGGACTCTGGGGTTCGAAATGACCGACCAAG
CGACGCCCAACCTGCCATCACGAGATTTCGATTCCACCGC
CGCCTTCTATGAAAGGTTGGGCTTCGGAATCGTTTTCCGG
GACGCCGGCTGGATGATCCTCCAGCGCGGGGATCTCATGC
TGGAGTTCTTCGCCCACCCCCATGGGCAAATATTATACGC
AAGGCGACAAGGTGCTGATGCCGCTGGCGATTCAGGTTCA
TCATGCCGTTTGTGATGGCTTCCATGTCGGCAGAATGCTT
AATGAATTACAACAGTTTTTATGCATGCGCCCAATACGCA
AACCGCCTCTCCCCGCGCGTTGGCCGATTCATTAATGCAG
CTGGCACGACAGGTTTCCCGACTGGAAAGCGGGCAGTGAG
CGCAACGCAATTAATGTGAGTTAGCTCACTCATTAGGCAC
CCCAGGCTTTACACTTTATGCTTCCGGCTCGTATGTTGTG
TGGAATTGTGAGCGGATAACAATTTCACACAGGAAACAGC
TATGACCATGATTACGCCAAGCGCGCAATTAACCCTCACT
AAAGGGAACAAAAGCTGGGTACCGAGCCCGCCTAATGAGC
GGGCTTTTTTTTCGTCGACGCTCTCCCTTATGCGACTCCT
GCATTAGGAAGCAGCCCAGTAGTAGGTTGAGGCCGTTGAG
CACCGCCGCCGCAAGGAATGGTGCATGCAAGGAGATGGCG
CCCAACAGTCCCCCGGCCACGGGGCCTGCCACCATACCCA
CGCCGAAACAAGCGCTCATGAGCCCGAAGTGGCGAGCCCG
ATCTTCCCCATCGGTGATGTCGGCGATATAGGCGCCAGCA
ACCGCACCTGTGGCGCCGGTGATGCCGGCCACGATGCGTC
CGGCGTAGAGGATCATCCTGTTTTGGCGGATGAGATAAGA
TTTTCAGCCTGATACAGATTAAATCAGAACGCAGAAGCGG
TCTGATAAAACAGAATTTGCCTGGCGGCAGTAGCGCGGTG
GTCCCACCTGACCCCATGCCGAACTCAGAAGTGAAACGCC
GTAGCGCCGATGGTAGTGTGGGGTCTCCCCATGCGAGAGT
AGGGAACTGCCAGGCATCAAATAAAACGAAAGGCTCAGTC
GAAAGACTGGGCCTTTCGTTTTATCTGTTGTTTGTCGGTG
AACGCTCTCCTGAGTAGGACAAATCCGCCGGGAGCGGATT
TGAACGTTGCGAAGCAACGGCCCGGAGGGTGGCGGGCAGG
ACGCCCGCCATAAACTGCCAGGCATCAAATTAAGCAGAAG
GCCATCCTGACGGATGGCCTTTTTGCGTTTCTACAAACTC
TCCATGGAAGGGCGAATTGATCCAGATGACGGTATCGATA
AGCTTGATATCAACAAGTTTGTACAAAAAAGCAGGCTCCC
CGGAATTGCCAGCTGGGGCGCCCTCTGGTAAGGTTGGGAA
GCCCTGCAAATGAAAACTCCCGAAGACTGCACCGGCCTGG
CGGACATCCGCGAGGCCATCGACCGGATCGACCTGGATAT
CGTCCAGGCCCTCGGCCGCCGCATGGACTACGTCAAGGCG
GCGTCGCGCTTCAAGGCCAGCGAGGCGGCGATTCCGGCGC
CCGAGCGGGTCGCCGCGATGCTCCCCGAGCGCGCCCGCTG
GGCCGAGGAAAACGGACTCGACGCGCCCTTCGTCGAGGGA
CTGTTCGCGCAGATCATCCACTGGTACATCGCCGAGCAGA
TCAAGTACTGGCGCCAGACACGGGGTGCCGCATGAGCCGG
CTGGCGCCCCTGAGCCAGTGCCTGCACGCCTTGCGCGGCA
CCTTCGAGCGCGCCATCGGCCAGGCGCAGGCGCTCGATCG
TCCGGTGCTGGTGGCGGCATCGTTCGAGATCGACCCATTG
GACCCGCTACAGGTATTCGGTGCCTGGGACGACCGGCAAA
CGCCCTGCCTGTACTGGGAACAGCCCGAGCTGGCGTTCTT
CGCCTGGGGCTGCGCCCTGGAGCTGCAAGGCCACGGCGAA
CAGCGCTTCGCCCGGATCGAGGAAAACTGGCAATTGCTCT
GCGCCGACGCCGTGGTCGAGGGCCCGCTGGCGCCGCGCCT
GTGCGGCGGATTCCGCTTCGATCCGCGCGGCCCGCGCGAG
GAACACTGGCAAGCCTTCGCCGATGCCAGCCTGATGCTCG
CCGGCATCACCGTGCTGCGCGAGGGCGAACGCTACCGGGT
ACTCTGCCAACACCTGGCCAAGCCCGGCGAAGATGCCCTG
GCCCTGGCCGCCTACCACTGCTCGGCGCTACTGCGCCTGA
GGCAGCCGGCCAGACGCCGGCCCTCGGGGCCGACCGCTGG
CGCGCAGGGCGACGCTTCGGCGCAGGAGCGCAGGCAATGG
GAAGCCAAGGTGAGCGACGCGGTAAGCAGTGTCCGCCAGG
GACGCTTCGGCAAGGTCGTGCTGGCCCGCACCCAGGCCCG
GCCTCTCGGCGACATCGAGCCGTGGCAGGTCATCGAACAC
CTGCGTCTGCAACATGCCGACGCCCAGCTGTTCGCCTGTC
GCCGCGGCAACGCCTGCTTCCTCGGCGCCTCCCCGGAACG
CCTGGTCCGCATTCGCGCCGGCGAGGCACTCACCCATGCC
CTGGCCGGGACCATCGCCCGCGGCGGCGATGCCCAGGAAG
ATGCGCGGCTCGGACAGGCCCTGCTGGACAGCGCCAAGGA
CAGGCACGAACACCAGTTGGTGGTGGAGGCGATCCGTACG
GCCCTGGAACCCTTCAGCGAGGTGCTGGAAATCCCCGATG
CGCCCGGCCTGAAACGACTGGCGCGAGTCCAGCACCTGAA
CACGCCGATCCGCGCCCGCCTCGCTGACGCAGGCGGCATC
CTGCGGCTGCTACAAGCGCTGCATCCGACCCCCGCGGTGG
GCGGCTACCCACGCAGCGCGGCGCTGGACTACATCCGCCA
GCACGAAGGGATGGACCGCGGCTGGTACGCCGCGCCGCTG
GGCTGGCTCGACGGCGAAGGCAACGGCGATTTCCTGGTGG
CGCTGCGCTCGGCCCTGCTCACGCCGGGCCGGGGCTACCT
GTTCGCCGGCTGCGGTCTGGTAGGCGATTCGGAACCGGCC
CACGAGTATCGCGAAACCTGCCTTAAGCTCAGTGCCATGC
GGGAAGCTCTATCCGCCATAGGCGGCCTGGACGAAGTGCC
CTTGCAGCGCGGCGTCGCCTAATAAGGTTGGGAAGCCCTG
CAAATGGAAGTTGTGGAAGTTCTGCACATGAATGGTGGTA
ACGGTGATAGCAGCTATGCGAATAATAGCCTGGTTCAACA
AAAAGTTATCCTGATGACCAAGCCGATCACCGAGCAGGCG
ATGATTGACCTGTACAGCAGCCTGTTTCCGGAAACCCTGT
GCATCGCGGATCTGGGTTGCAGCCTGGGCGCGAACACCTT
CCTGGTGGTTAGCCAACTGGTGAAGATTGTTGAGAAAGAG
CGTAAGAAACACGGTTTTAAGAGCCCGGAGTTCTATTTTC
ACTTCAACGACCTGCCGGGTAACGATTTTAACACCCTGTT
CCAGAGCCTGGGCGCGTTTCAAGAGGACCTGCGTAAACAC
ATCGGCGAGAGCTTCGGCCCGTGCTTCTTTAGCGGTGTGC
CGGGCAGCTTTTACACCCGTCTGTTCCCGAGCAAGAGCCT
GCACTTCGTGTACAGCAGCTATAGCCTGATGTGGCTGAGC
CAGGTTCCGAACGGTATCGAGAACAACAAAGGCAACATTT
ATATGGCGCGTACCAGCCCGCTGAGCGTGATCAAGGCGTA
CTACAAGCAATACGAAATCGACTTCAGCAACTTCCTGAAG
TATCGTAGCGAGGAACTGATGAAGGGTGGCAAAATGGTTC
TGACCCTGCTGGGTCGTGAGAGCGAAGATCCGACCAGCAA
GGAGTGCTGCTACATCTGGGAACTGCTGGCGATGGCGCTG
AACAAACTGGTGGAGGAAGGCCTGATCAAAGAGGAGAAAG
TTGACGCGTTTAACATTCCGCAATACACCCCGAGCCCGGC
GGAAGTGAAGTATATCGTTGAGAAAGAAGGCAGCTTCACC
ATTAACCGTCTGGAAACCAGCCGTGTGCACTGGAACGCGA
GCAACAACGAGAAGAACGGTGGCTACAACGTTAGCCGTTG
CATGCGTGCGGTGGCGGAGCCGCTGCTGGTTAGCCACTTT
GACAAGGAACTGATGGATCTGGTGTTCCACAAATATGAGG
AAATTGTTAGCGATTGCATGAGCAAGGAGAACACCGAGTT
TATCAATGTTATTATTAGCCTGACCAAAATCAATTAACAC
CCAGCTTTCTTGTACAAAGTGGTTGATATCTACCCTTACG
ATGTTCCTGATTACGCATGAGGATCCGGTGATTGATTGAG
CAAGCTTTATGCTTGTAAACCGTTTTGTGAAAAAATTTTT
AAAATAAAAAAGGGGACCTCTAGGGTCCCCAATTAATTAG
TAATATAATCTATTAAAGGTCATTCAAAAGGTCATCCACC
GGATCAATTCCCCTGCTCGCGCAGGCTGGGTGCCAAGCTC
TCGGGTAACATCAAGGCCCGATCCTTGGAGCCCTTGCCCT
CCCGCACGATGATCGTGCCGTGATCGAAATCCAGATCCTT
GACCCGCAGTTGCAAACCCTCACTGATCCGCTCCAATTCG
CCCTATAGTGAGTCGTATTACGCGCGCTCACTGGCCGTCG
TTTTACAACGTCGTGACTGGGAAAACCCTGGCGTTACCCA
ACTTAATCGCCTTGCAGCACATCCCCCTTTCGCCAGCTGG
CGTAATAGCGAAGAGGCCCGCACCGATCGCCCTTCCCAAC
AGTTGCGCAGCCTGAATGGCGAATGGAAATTGTAAGCGTT
AATATTTTGTTAAAATTCGCGTTAAATTTTTGTTAAATCA
GCTCATTTTTTAACCAATAGGCCGTACTGCGATGAGTGGC
AGGGCGGGGCGTAATTTTTTTAAGGCAGTTATTGGTGCCC
TTAAACGCCTGGTTGCTACGCCTGAATAAGTGATAATAAG
CGGATGAATGGCAGAAATTCGAAAGCAAATTCGACCCGGT
CGTCGGTTCAGGGCAGGGTCGTTAAATAGCCGCTTATGTC
TATTGCTGGTTTACCGGTTTATTGACTACCGGAAGCAGTG
TGACCGTGTGCTTCTCAAATGCCTGAGGCCAGTTTGCTCA
GGCTCTCCCCGTGGAGGTAATAATTGACGATATGATCATT
TATTCTGCCTCCCAGAGCCTGATAAAAACGGTGAATCCGT
TAGCGAGGTGCCGCCGGCTTCCATTCAGGTCGAGGTGGCC
CGGCTCCATGCACCGCGACGCAACGCGGGGAGGCAGACAA
GGTATAGGGCGGCGAGGCGGCTACAGCCGATAGTCTGGAA
CAGCGCACTTACGGGTTGCTGCGCAACCCAAGTGCTACCG
GCGCGGCAGCGTGACCCGTGTCGGCGGCTCCAACGGCTCG
CCATCGTCCAGAAAACACGGCTCATCGGGCATCGGCAGGC
GCTGCTGCCCGCGCCGTTCCCATTCCTCCGTTTCGGTCAA
GGCTGGCAGGTCTGGTTCCATGCCCGGAATGCCGGGCTGG
CTGGGCGGCTCCTCGCCGGGGCCGGTCGGTAGTTGCTGCT
CGCCCGGATACAGGGTCGGGATGCGGCGCAGGTCGCCATG
CCCCAACAGCGATTCGTCCTGGTCGTCGTGATCAACCACC
ACGGCGGCACTGAACACCGACAGGCGCAACTGGTCGCGGG
GCTGGCCCCACGCCACGCGGTCATTGACCACGTAGGCCGA
CACGGTGCCGGGGCCGTTGAGCTTCACGACGGAGATCCAG
CGCTCGGCCACCAAGTCCTTGACTGCGTATTGGACCGTCC
GCAAAGAACGTCCGATGAGCTTGGAAAGTGTCTTCTGGCT
GACCACCACGGCGTTCTGGTGGCCCATCTGCGCCACGAGG
TGATGCAGCAGCATTGCCGCCGTGGGTTTCCTCGCAATAA
GCCCGGCCCACGCCTCATGCGCTTTGCGTTCCGTTTGCAC
CCAGTGACCGGGCTTGTTCTTGGCTTGAATGCCGATTTCT
CTGGACTGCGTGGCCATGCTTATCTCCATGCGGTAGGGGT
GCCGCACGGTTGCGGCACCATGCGCAATCAGCTGCAACTT
TTCGGCAGCGCGACAACAATTATGCGTTGCGTAAAAGTGG
CAGTCAATTACAGATTTTCTTTAACCTACGCAATGAGCTA
TTGCGGGGGGTGCCGCAATGAGCTGTTGCGTACCCCCCTT
TTTTAAGTTGTTGATTTTTAAGTCTTTCGCATTTCGCCCT
ATATCTAGTTCTTTGGTGCCCAAAGAAGGGCACCCCTGCG
GGGTTCCCCCACGCCTTCGGCGCGGCTCCCCCTCCGGCAA
AAAGTGGCCCCTCCGGGGCTTGTTGATCGACTGCGCGGCC
TTCGGCCTTGCCCAAGGTGGCGCTGCCCCCTTGGAACCCC
CGCACTCGCCGCCGTGAGGCTCGGGGGGCAGGCGGGCGGG
CTTCGCCCTTCGACTGCCCCCACTCGCATAGGCTTGGGTC
GTTCCAGGCGCGTCAAGGCCAAGCCGCTGCGCGGTCGCTG
CGCGAGCCTTGACCCGCCTTCCACTTGGTGTCCAACCGGC
AAGCGAAGCGCGCAGGCCGCAGGCCGGAGGCTTTTCCCCA
GAGAAAATTAAAAAAATTGATGGGGCAAGGCCGCAGGCCG
CGCAGTTGGAGCCGGTGGGTATGTGGTCGAAGGCTGGGTA
GCCGGTGGGCAATCCCTGTGGTCAAGCTCGTGGGCAGGCG
CAGCCTGTCCATCAGCTTGTCCAGCAGGGTTGTCCACGGG
CCGAGCGAAGCGAGCCAGCCGGTGGCCGCTCGCGGCCATC
GTCCACATATCCACGGGCTGGCAAGGGAGCGCAGCGACCG
CGCAGGGCGAAGCCCGGAGAGCAAGCCCGTAGGGCGCCGC
AGCCGCCGTAGGCGGTCACGACTTTGCGAAGCAAAGTCTA
GTGAGTATACTCAAGCATTGAGTGGCCCGCCGGAGGCACC
GCCTTGCGCTGCCCCCGTCGAGCCGGTTGGACACCAAAAG
GGAGGGGCAGGCATGGCGGCATACGCGATCATGCGATGCA
AGAAGCTGGCGAAAAT

SEQ ID NOs: 14-48 are provided in the sequence listing filed herewith, which is incorporated by reference as if expressed in its entirety herein. SEQ ID NOS: 14-48 provide genome sequences of bacteria isolated from Dog53 “Dog53 isolates”.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An engineered bacterium or population thereof, wherein the engineered bacterium comprises:
one or more polynucleotides that each encode one or more enzymes capable of catalyzing one or more reactions to produce a volatile compound, one or more polypeptides produced therefrom, or both
and wherein the engineered bacterium is of a strain that is a commensal bacterium strain found in the oral cavity of a mammal, optionally a canine or a feline.

2. The engineered bacterium or population thereof of aspect 1, wherein the one or more enzymes is/are
a. a methyltransferase that catalyzes the production of methyl salicylate from salicylic acid;
b. an isochorismate synthase;
c. an ischorisomate-pyruvate lyase;
d. is an alcohol acetyl transferase that is capable of condensing isoamyl alcohol and acetyl CoA into isoamyl acetate; or
e. any combination thereof.

3. The engineered bacterium or population thereof of any one of aspects 1-2, wherein the one or more polynucleotides each encode a methyltransferase that catalyzes the production of methyl salicylate from salicylic acid, an isochorismate synthase, an ischorisomate-pyruvate lyase, or any combination thereof.

4. The engineered bacterium or population thereof of any one of aspects 1-3, wherein the volatile compound produced has a mint odor.

5. The engineered bacterium or population thereof of any one of aspects 1-4, wherein the one or more polynucleotides each comprise a PCHBA gene or mRNA, a BSMT1 gene or mRNA, or any combination thereof.

6. The engineered bacterium or population thereof any one of aspects 1-5, wherein at least one of the one or more polynucleotides encodes an alcohol acetyl transferase that is capable of condensing isoamyl alcohol and acetyl CoA into isoamyl acetate.

7. The engineered bacterium or population thereof any one of aspects 1-6, wherein the engineered bacterium or population thereof produces a fruity odor.

8. The engineered bacterium or population thereof of aspect 7, wherein the fruity odor is a banana odor or a pear odor.

9. The engineered bacterium or population thereof any one of aspects 1-8, wherein at least one of the one or more polynucleotides comprises an ATF gene or mRNA, an BAT2 gene or mRNA, a THI3 gene or mRNA, or any combination thereof.

10. The engineered bacterium or population thereof of aspect 9, wherein the ATF gene or mRNA is an ATF1 gene or mRNA or an ATF2 gene or mRNA.

11. The engineered bacterium or population thereof any one of aspects 1-10, wherein one or more of the one or more polynucleotides each comprise a gene or mRNA independently selected from a BSTM1 gene, a BSTM1 mRNA, an ATF gene, an ATF mRNA, a PCHBA gene, a PCHBA mRNA, a BAT2 gene, a BAT2 mRNA, a THI3 gene, a THI3 mRNA, or any combination thereof.

12. The engineered bacterium or population thereof of aspect 11, wherein the ATF gene or ATF mRNA is an ATF1 gene or mRNA or an ATF2 gene or mRNA.

13. The engineered bacterium or population thereof of aspect 11, wherein
a. the BSTM1 gene or mRNA has a polynucleotide sequence that is about 90-100 percent identical to any one of SEQ ID NO: 2, 8, 11, or 13;
b. the PCHBA gene or mRNA has a polynucleotide sequence that is about 90-100 percent identical to SEQ ID NO: 1 or 10;
c. the ATF gene or mRNA has a polynucleotide sequence that is about 90-100 percent identical to SEQ ID NO: 49 or 51;
d. the BAT2 gene or mRNA has a polynucleotide sequence that is about 90-100 percent identical to SEQ ID NO: 53;
e. the THI3 gene or mRNA has a polynucleotide sequence that is about 90-100 percent identical to SEQ ID NO: 55

14. The engineered bacterium of any one of aspects 1-13, wherein one or more of the one or more polynucleotides has a sequence that is about 40-100% identical to any one of SEQ ID NOs.: 1, 2, 8, 10-11, 13, 49, 51, 53, or 55 or any region therein of at least 20 nucleotides.

15. The engineered bacterium of any one of aspects 1-14, wherein one or more of the one or more polynucleotides encodes a polypeptide having a sequence that is about 40 to 100% identical to any one of SEQ ID NOS.: 3-7, 9, 50, 52, 54, or 56.

16. The engineered bacterium or population thereof of any one of aspects 1-15, further comprising one or more expression vectors, wherein the one or more expression vectors comprise the one or more polynucleotides.

17. The engineered bacterium or population thereof any one of aspects 1-16, wherein the engineered bacterium is from genus selected from Escherichia, Pseudomonas, Brevundimonas, Xanthomonas, Deinococcus, or Pedobacter.

18. The engineered bacterium or population thereof any one of aspects 1-17, wherein the engineered bacterium is an isolate of a commensal canine oral bacterium or population thereof.

19. The engineered bacterium or population thereof any one of aspects 1-18, wherein the commensal canine oral bacterium or population thereof is of the genus Pseudomonas.

20. The engineered bacterium or population thereof any one of aspects 1-19, wherein the engineered bacterium is an isolated commensal canine oral bacterium or population thereof comprising a genome that comprises a sequence that is 90-100 percent identical to any one or more of SEQ ID NOs: 12 or 14-48.

21. An expression vector capable of expressing a BSTM1 polypeptide, a PCHB polypeptide, a PCHA polypeptide, an ATF1 polypeptide, an ATF2 polypeptide, BAT2 polypeptide, a THI3 polypeptide or any combination thereof comprising:
a. a BSTM1 polynucleotide comprising a sequence that is about 90-100 percent identical to any one of SEQ ID NO: 2, 8, 11, or 13;
b. a PCHBA polynucleotide comprising a sequence that is about 90-100 percent identical to SEQ ID NO: 1 or 10;
c. an ATF polynucleotide comprising a sequence that is about 90-100 percent identical to SEQ ID NO: 49 or 51;
d. a BAT2 polynucleotide comprising a sequence that is about 90-100 percent identical to SEQ ID NO: 53;
e. a THI3 polynucleotide comprising a sequence that is about 90-100 percent identical to SEQ ID NO: 55;
f. or any combination thereof.

22. The expression vector of aspect 21, wherein (a), (b), (c), (d), (e), or (0 is/are operatively coupled one or more regulatory elements.

23. The expression vector of any one of aspects 21-22, wherein the expression vector comprises a sequence that is about 90-100 percent identical to SEQ ID NO: 13.

24. A formulation comprising: an engineered bacterium of any one of aspects 1-20, an expression vector of any one of aspects 21-23, or both, optionally wherein the engineered bacterium comprises the expression vector and a liquid, semi-solid, or solid carrier.

25. The formulation of aspect 24, wherein the formulation is a foodstuff suitable for a mammal.

26. The formulation of any one of aspects 24-25, wherein the mammal is a non-human animal.

27. The formulation of any one of aspects 24-26, wherein the mammal is a canine or a feline.

28. The formulation of any one of aspects 24-27, wherein the formulation is a liquid solution or semi-solid suitable for administration to the oral cavity of a mammal.

29. The formulation of any one of aspects 24-28, wherein the mammal is a non-human animal.

30. The formulation of any one of aspects 24-29, wherein the mammal is a canine or a feline.

31. The formulation of any one of aspects 24-30, wherein the formulation is a formed object that is configured to be chewed on by a mammal.

32. The formulation of aspect 31, wherein the mammal is a non-human animal.

33. The formulation of any one of aspects 31-32, wherein the mammal is a canine or a feline.

34. A method of improving the breath of a mammal, the method comprising:

administering an engineered bacterium or population thereof of any one of aspects 1-20, an expression vector of any one of aspects 21-23, or both, or a formulation thereof to the mammal.

35. The method of aspect 34, further comprising allowing the engineered bacterium or population thereof to produce a volatile compound.

36. The method of any one of aspects 34-35, wherein the mammal is a non-human animal.

37. The method of any one of aspects 34-36, wherein the mammal is canine or a feline.

38. An isolated commensal canine oral bacterium or population thereof comprising: a genome comprising a sequence that is 90-100 percent identical to any one or more of SEQ ID NOs: 12 or 14-48.

What is claimed is:

1. An engineered bacterium or population thereof, wherein the engineered bacterium comprises:
one or more polynucleotides that each encode one or more enzymes capable of catalyzing one or more reactions to produce a volatile compound, one or more polypeptides produced therefrom, or both
and wherein the engineered bacterium is of a strain that is a commensal bacterium strain found in the oral cavity of a mammal, optionally a canine or a feline.

2. The engineered bacterium or population thereof of claim 1, wherein the one or more enzymes is/are
a. a methyltransferase that catalyzes the production of methyl salicylate from salicylic acid;
b. an isochorismate synthase;
c. an ischorisomate-pyruvate lyase;
d. is an alcohol acetyl transferase that is capable of condensing isoamyl alcohol and acetyl CoA into isoamyl acetate; or
e. any combination thereof.

3. The engineered bacterium or population thereof of claim 1, wherein the one or more polynucleotides each encode a methyltransferase that catalyzes the production of methyl salicylate from salicylic acid, an isochorismate synthase, an ischorisomate-pyruvate lyase, or any combination thereof.

4. The engineered bacterium or population thereof of claim 3, wherein the volatile compound produced has a mint odor.

5. The engineered bacterium or population thereof of claim 3, wherein the one or more polynucleotides each comprise a PCHBA gene or mRNA, a BSMT1 gene or mRNA, or any combination thereof.

6. The engineered bacterium or population thereof of claim 1, wherein at least one of the one or more polynucleotides encodes an alcohol acetyl transferase that is capable of condensing isoamyl alcohol and acetyl CoA into isoamyl acetate.

7. The engineered bacterium or population thereof of claim 6, wherein the engineered bacterium or population thereof produces a fruity odor.

8. The engineered bacterium or population thereof of claim 7, wherein the fruity odor is a banana odor or a pear odor.

9. The engineered bacterium or population thereof of claim 6, wherein at least one of the one or more polynucleotides comprises an ATF gene or mRNA, an BAT2 gene or mRNA, a THI3 gene or mRNA, or any combination thereof.

10. The engineered bacterium or population thereof of claim 9, wherein the ATF gene or mRNA is an ATF1 gene or mRNA or an ATF2 gene or mRNA.

11. The engineered bacterium or population thereof of claim 1, wherein one or more of the one or more polynucleotides each comprise a gene or mRNA independently selected from a BSTM1 gene, a BSTM1 mRNA, an ATF gene, an ATF mRNA, a PCHBA gene, a PCHBA mRNA, a BAT2 gene, a BAT2 mRNA, a THI3 gene, a THI3 mRNA, or any combination thereof.

12. The engineered bacterium or population thereof of claim 11, wherein the ATF gene or ATF mRNA is an ATF1 gene or mRNA or an ATF2 gene or mRNA.

13. The engineered bacterium or population thereof of claim 11, wherein
a. the BSTM1 gene or mRNA has a polynucleotide sequence that is about 90-100 percent identical to any one of SEQ ID NO: 2, 8, 11, or 13;
b. the PCHBA gene or mRNA has a polynucleotide sequence that is about 90-100 percent identical to SEQ ID NO: 1 or 10;
c. the ATF gene or mRNA has a polynucleotide sequence that is about 90-100 percent identical to SEQ ID NO: 49 or 51;
d. the BAT2 gene or mRNA has a polynucleotide sequence that is about 90-100 percent identical to SEQ ID NO: 53;
e. the THI3 gene or mRNA has a polynucleotide sequence that is about 90-100 percent identical to SEQ ID NO: 55

14. The engineered bacterium of claim 1, wherein one or more of the one or more polynucleotides has a sequence that is about 40-100% identical to any one of SEQ ID NOs.:
1, 2, 8, 10-11, 13, 49, 51, 53, or 55 or any region therein of at least 20 nucleotides.

15. The engineered bacterium of claim 1, wherein one or more of the one or more polynucleotides encodes a polypeptide having a sequence that is about 40 to 100% identical to any one of SEQ ID NOS.: 3-7, 9, 50, 52, 54, or 56.

16. The engineered bacterium or population thereof of claim 1, further comprising one or more expression vectors, wherein the one or more expression vectors comprise the one or more polynucleotides.

17. The engineered bacterium or population thereof of claim 1, wherein the engineered bacterium is from genus selected from Escherichia, Pseudomonas, Brevundimonas, Xanthomonas, Deinococcus, or Pedobacter.

18. The engineered bacterium or population thereof of claim 1, wherein the engineered bacterium is an isolate of a commensal canine oral bacterium or population thereof.

19. The engineered bacterium or population thereof of claim 18, wherein the commensal canine oral bacterium or population thereof is of the genus Pseudomonas.

20. The engineered bacterium or population thereof of claim 1, wherein the engineered bacterium is an isolated commensal canine oral bacterium or population thereof comprising a genome that comprises a sequence that is 90-100 percent identical to any one or more of SEQ ID NOs: 12 or 14-48.

21. An expression vector capable of expressing a BSTM1 polypeptide, a PCHB polypeptide, a PCHA polypeptide, an ATF1 polypeptide, an ATF2 polypeptide, BAT2 polypeptide, a THI3 polypeptide or any combination thereof comprising:
a. a BSTM1 polynucleotide comprising a sequence that is about 90-100 percent identical to any one of SEQ ID NO: 2, 8, 11, or 13;
b. a PCHBA polynucleotide comprising a sequence that is about 90-100 percent identical to SEQ ID NO: 1 or 10;
c. an ATF polynucleotide comprising a sequence that is about 90-100 percent identical to SEQ ID NO: 49 or 51;
d. a BAT2 polynucleotide comprising a sequence that is about 90-100 percent identical to SEQ ID NO: 53;
e. a THI3 polynucleotide comprising a sequence that is about 90-100 percent identical to SEQ ID NO: 55;
f. or any combination thereof.

22. The expression vector of claim 21, wherein (a), (b), (c), (d), (e), or (0 is/are operatively coupled one or more regulatory elements.

23. The expression vector of claim 22, wherein the expression vector comprises a sequence that is about 90-100 percent identical to SEQ ID NO: 13.

24. A formulation comprising:
an engineered bacterium of any one of claims 1-20, an expression vector of any one of claims 21-23, or both, optionally wherein the engineered bacterium comprises the expression vector and
a liquid, semi-solid, or solid carrier.

25. The formulation of claim 24, wherein the formulation is a foodstuff suitable for a mammal.

26. The formulation of claim 25, wherein the mammal is a non-human animal.

27. The formulation of claim 26, wherein the mammal is a canine or a feline.

28. The formulation of claim 24, wherein the formulation is a liquid solution or semi-solid suitable for administration to the oral cavity of a mammal.

29. The formulation of claim 28, wherein the mammal is a non-human animal.

30. The formulation of claim 29, wherein the mammal is a canine or a feline.

31. The formulation of claim 24, wherein the formulation is a formed object that is configured to be chewed on by a mammal.

32. The formulation of claim 31, wherein the mammal is a non-human animal.

33. The formulation of claim 32, wherein the mammal is a canine or a feline.

34. A method of improving the breath of a mammal, the method comprising:
administering an engineered bacterium or population thereof of any one of claims 1-20, an expression vector of any one of claims 21-23, or both, or a formulation thereof to the mammal.

35. The method of claim 34, further comprising allowing the engineered bacterium or population thereof to produce a volatile compound.

36. The method of claim 35, wherein the mammal is a non-human animal.

37. The method of claim 36, wherein the mammal is canine or a feline.

38. An isolated commensal canine oral bacterium or population thereof comprising:
a genome comprising a sequence that is 90-100 percent identical to any one or more of SEQ ID NOs: 12 or 14-48.
